(19) United States
(12) Patent Application Publication
(10) Pub. No.: US 2008/0214529 A1
(43) Pub. Date: Sep. 4, 2008

(54) SATURATED FUSED [1,2-b]PYRIDAZINONE COMPOUNDS

(76) Inventors: Frank Ruebsam, San Diego, CA (US); Zhongxiang Sun, San Diego, CA (US); Benjamin Ayida, Chula Vista, CA (US); Yuefen Zhou, San Diego, CA (US); Alan X. Xiang, San Diego, CA (US)

Correspondence Address: CONNOLLY BOVE LODGE & HUTZ LLP, 1875 EYE STREET, N.W., SUITE 1100, WASHINGTON, DC, 20036, US

(21) Appl. No.: 11/955,193

(22) Filed: Dec. 12, 2007

Related U.S. Application Data
(60) Provisional application No. 60/874,252, filed on Dec. 12, 2006.

(51) Int. Cl. A61K 31/5415; C07D 417/04; C12N 7/06; A61P 31/12

(52) U.S. Cl. 514/223.5; 544/12; 514/223.2; 435/238

(57) ABSTRACT

The invention is directed to saturated fused [1,2-b]pyridazinone compounds and pharmaceutical compositions containing such compounds that are useful in treating infections by hepatitis C virus.

This application claims the benefit of U.S. Provisional Application No. 60/874,252, filed Dec. 12, 2006.

### FIELD OF THE INVENTION

The invention is directed to saturated fused [1,2-b]pyridazinone compounds and pharmaceutical compositions containing such compounds that are useful in treating infections by hepatitis C virus.

### BACKGROUND OF THE INVENTION

Hepatitis C is a major health problem world-wide. The World Health Organization estimates that 170 million people are chronic carriers of the hepatitis C virus (HCV), with 4 million carriers in the United States alone. In the United States, HCV infection accounts for 40% of chronic liver disease and HCV disease is the most common cause for liver transplantation. HCV infection leads to a chronic infection and about 70% of persons infected will develop chronic histological changes in the liver (chronic hepatitis) with a 10-40% risk of cirrhosis and an estimated 4% lifetime risk of hepatocellular carcinoma. The CDC estimates that each year in the United States there are 35,000 new cases of HCV infection and approximately ten thousand deaths attributed to HCV disease.

The current standard of care is a pegylated interferon/ribavirin combination at a cost of approximately $31,000/year. These drugs have difficult dosing problems and side-effects that preclude their use in almost half of diagnosed patients. Pegylated interferon treatment is associated with menacing flu-like symptoms, irritability, inability to concentrate, suicidal ideation, and leukocytopenia. Ribavirin is associated with hemolytic anemia and birth defects.

The overall response to this standard therapy is low; approximately one third of patients do not respond. Of those who do respond, a large fraction relapses within six months of completing 6-12 months of therapy. As a consequence, the long-term response rate for all patients entering treatment is only about 50%. The relatively low response rate and the significant side-effects of current therapy anti-HCV drug treatments, coupled with the negative long term effects of chronic HCV infection, result in a continuing medical need for improved therapy. Antiviral pharmaceuticals to treat RNA virus diseases like HCV are few, and as described above are often associated with multiple adverse effects.

A number of recent publications have described NS5B inhibitors useful in the treatment of hepatitis C infection. See, e.g., U.S. Patent Application Publication No. US 2006/0189602 (disclosing certain pyridazinones); U.S. Patent Application Publication No. US 2006/0252785 (disclosing selected heterocyclics); and International Publication Nos. WO 03/059356, WO 2002/098424, and WO 01/85172 (each describing a particular class of substituted thiadiazines).

While there are, in some cases, medicines available to reduce disease symptoms, there are few drugs to effectively inhibit replication of the underlying virus. The significance and prevalence of RNA virus diseases, including but not limited to chronic infection by the hepatitis C virus, and coupled with the limited availability and effectiveness of current antiviral pharmaceuticals, have created a compelling and continuing need for new pharmaceuticals to treat these diseases.

### SUMMARY OF THE INVENTION

The present invention describes novel saturated fused [1,2-b]pyridazinone compounds, pharmaceutically acceptable salts, and pharmaceutically acceptable solvates thereof, which are useful in treating or preventing a hepatitis C virus infection in a patient in need thereof comprising administering to the patient a therapeutically or prophylactically effective amount of a saturated fused [1,2-b]pyridazinone compound.

In a general aspect, the invention relates to compounds of Formula I

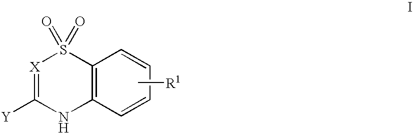

wherein

### X is N or CR³,

### Y is

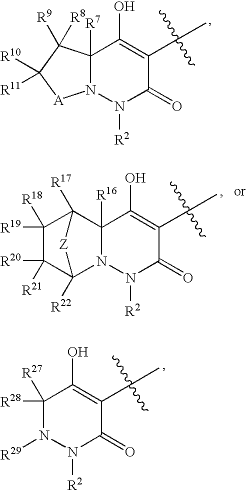

wherein

### A is —CR¹²R¹³— or —CR¹²R¹³—CR¹⁴R¹⁵—,

### Z is —CR²³R²⁴— or —CR²³R²⁴—CR²⁵R²⁶—,

R¹ is H, halo, nitro, —CHR⁴—S(O)₂R⁵, —NR⁵R⁶, —NR⁴S(O)₂R⁵, or —NR⁴S(O)₂NR⁵R⁶, wherein R⁴, R⁵, and R⁶ are independently H, C₁-C₆ alkyl, C₃-C₈ cycloalkyl, aryl, or heterocyclyl, or R⁴ and R⁵ or R⁵ and R⁶ combine with the atom(s) to which they are attached to form a 5- or 6-membered heterocyclyl ring,
R² is C₁-C₆ alkyl, C₃-C₈ cycloalkyl, —C₁-C₆ alkylene(C₃-C₈ cycloalkyl), —C₁-C₆ alkylene(aryl), or —C₁-C₆ alkylene(heterocyclyl),
R³ is H, halo, or C₁-C₆ alkyl,
R⁷ is H, C₁-C₆ alkyl, or C₃-C₈ cycloalkyl, or R⁷ and R⁸ or R⁷ and R⁹ combine to form a 3-membered ring,
R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently H, C₁-C₆ alkyl, or halo, or
R¹⁰ and R¹² or R¹⁰ and R¹³, or R¹¹ and R¹² or R¹¹ and R¹³ combine to form a 5-membered ring, or
R²⁷ and R²⁸ combine to form a 3- to 5-membered ring,
R²⁹ is H or C₁-C₆ alkyl,
wherein the above alkyl, alkylene, aryl, cycloalkyl, or heterocyclyl moieties provided in R¹ through R²⁹ are each optionally and independently substituted by 1-3 substituents selected from
alkylamine,
amino,
aryl, cycloalkyl, heterocyclyl,
C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms,
carboxyl,
cyano,
halo,
hydroxy,
nitro,
—C(O)OH, —C(O)₂—(C₁-C₆ alkyl), —C(O)₂—(C₃-C₈ cycloalkyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkylene), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkyl)cycloalkyl,
   wherein each of the above optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents, or a pharmaceutically acceptable salt, hydrate, tautomer or stereoisomer thereof.

In one embodiment, the invention relates to compounds of Formula I wherein R¹ is —NR⁴S(O)₂R⁵, wherein R⁴ and R⁵ are independently H or C₁-C₆ alkyl.

In another embodiment, the invention relates to compounds of Formula I wherein R¹ is selected from

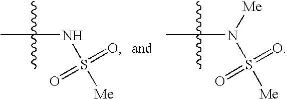

In one embodiment, the invention relates to compounds of Formula I wherein R² is selected from

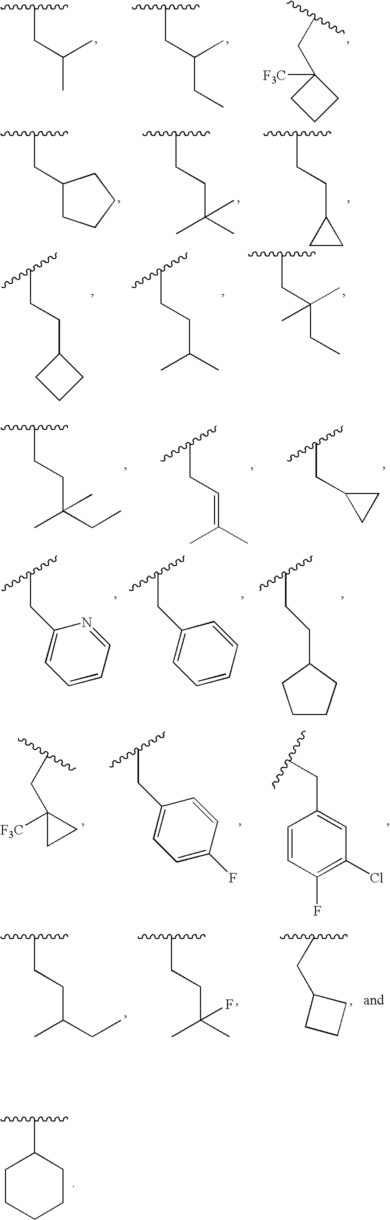

In another embodiment, the invention relates to compounds of Formula I wherein R² is selected from

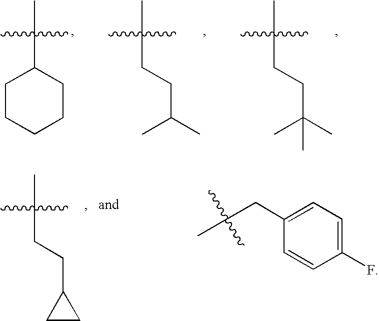

In a further embodiment, the invention relates to compounds of Formula I wherein R² is selected from

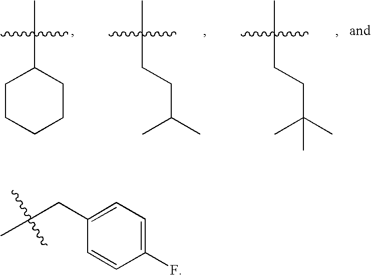

In one embodiment, the invention relates to compounds of Formula I wherein R³ is selected from

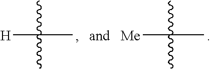

In one embodiment, the invention relates to compounds of Formula I wherein X is N.

In one embodiment, the invention relates to compounds of Formula I wherein R⁷ is selected from H or C₁-C₆ alkyl.

In a further embodiment, the invention relates to compounds of Formula I wherein R⁷ is selected from

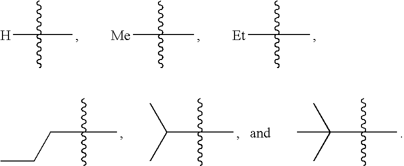

In another embodiment, the invention relates to compounds of Formula I wherein R⁷ is selected from

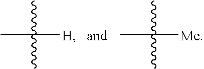

In one embodiment, the invention relates to compounds of Formula I wherein Y is

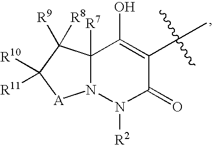

wherein A is —CR¹²R¹³—, and R², R⁷, R⁸, R⁹, R¹⁰, and R¹¹ are defined as above.

In one embodiment, the invention relates to compounds of Formula I wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from

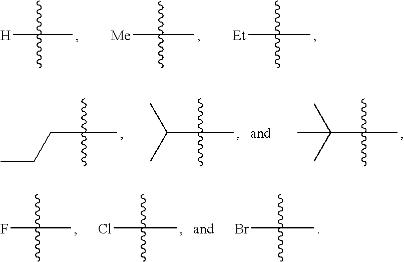

In another embodiment, the invention relates to compounds of Formula I wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷ and R²⁸ are independently selected from

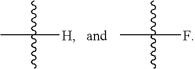

In a further embodiment, the invention relates to compounds of Formula I wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are H.

In one embodiment, the invention relates to compounds of Formula I wherein R²⁹ is methyl.

In one embodiment, the invention relates to compounds selected from

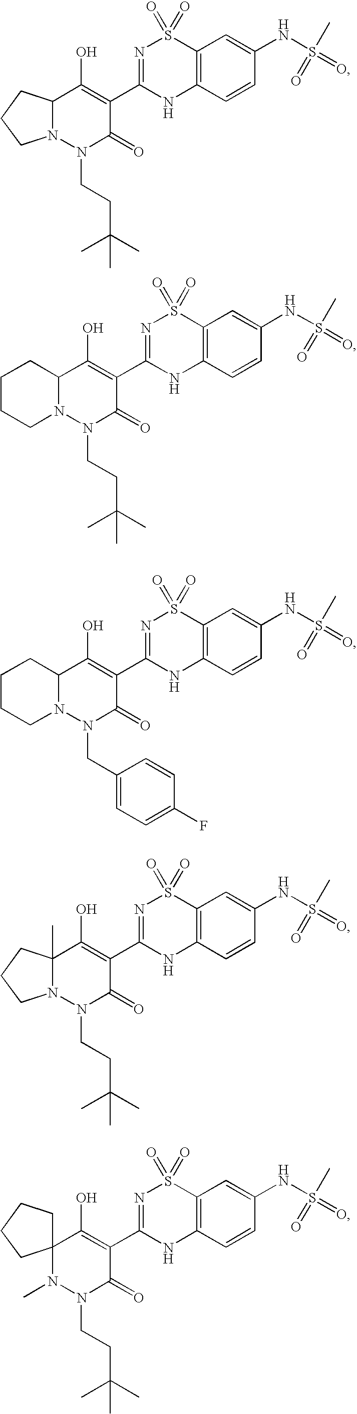

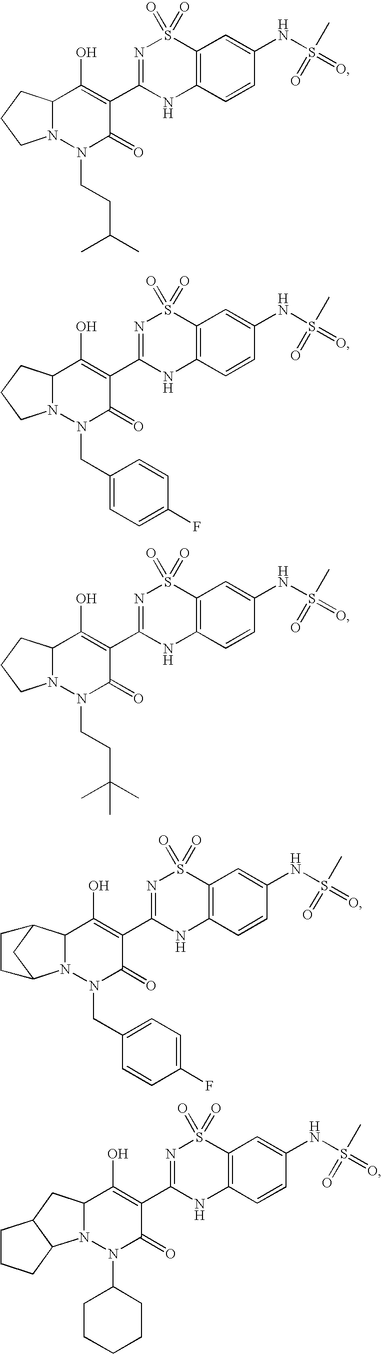

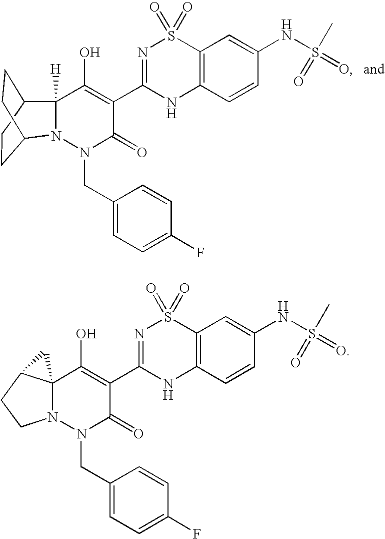

The invention is also directed to pharmaceutically acceptable salts and pharmaceutically acceptable solvates of the compounds of Formula I. Advantageous methods of making the compounds of Formula I are also described.

In one aspect, the invention encompasses a method for treating or preventing hepatitis C virus infection in a mammal in need thereof, preferably in a human in need thereof, comprising administering to the patient a therapeutically or prophylactically effective amount of a Formula I compound. In one embodiment, the invention encompasses a method for treating or preventing hepatitis C virus infection by administering to a patient in need thereof a therapeutically or prophylactically effective amount of a Formula I compound that is an inhibitor of HCV NS5B polymerase.

In another aspect, the invention encompasses a method for treating or preventing hepatitis C virus infection in a patient in need thereof, comprising administering to the patient a therapeutically or prophylactically effective amount of a compound of Formula I and a pharmaceutically acceptable excipient, carrier, or vehicle.

In another aspect, the invention encompasses a method for treating or preventing hepatitis C virus infection in a patient in need thereof, comprising administering to the patient a therapeutically or prophylactically effective amount of a compound of Formula I and an additional therapeutic agent, preferably an additional antiviral agent or an immunomodulatory agent.

### DETAILED DESCRIPTION OF THE INVENTION

Where the following terms are used in this specification, they are used as defined below:

The terms “comprising,” “having” and “including” are used herein in their open, non-limiting sense.

The term “alkyl”, as used herein, unless otherwise indicated, includes saturated monovalent hydrocarbon radicals having straight, branched, or cyclic moieties (including fused and bridged bicyclic and spirocyclic moieties), or a combination of the foregoing moieties. For an alkyl group to have cyclic moieties, the group must have at least three carbon atoms.

The term “alkylene”, as used herein, unless otherwise indicated, includes a divalent radical derived from alkyl, as exemplified by —CH₂CH₂CH₂CH₂—.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “Me” means methyl, “Et” means ethyl, and “Ac” means acetyl.

The term “cycloalkyl”, as used herein, unless otherwise indicated refers to a non-aromatic, saturated or partially saturated, monocyclic or fused, spiro or unfused bicyclic or tricyclic hydrocarbon referred to herein containing a total of from 3 to 10 carbon atoms, preferably 5-8 ring carbon atoms. Exemplary cycloalkyls include monocyclic rings having from 3-7, preferably 3-6, carbon atoms, such as cyclopropyl, cyclobutyl, cyclopentyl, cyclohexyl, cycloheptyl and the like. Illustrative examples of cycloalkyl are derived from, but not limited to, the following:

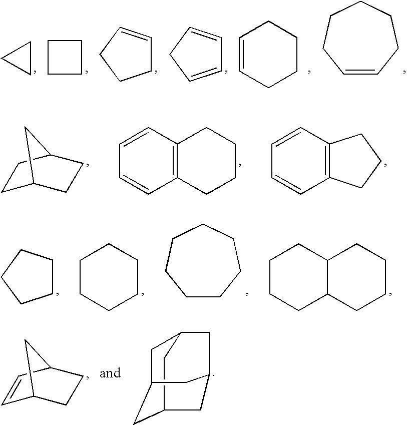

The term “aryl”, as used herein, unless otherwise indicated, includes an organic radical derived from an aromatic hydrocarbon by removal of one hydrogen, such as phenyl or naphthyl.

The term “heterocyclic” or “heterocyclyl”, as used herein, unless otherwise indicated, includes aromatic (e.g., heteroaryls) and non-aromatic heterocyclic groups containing one to four heteroatoms each selected from O, S and N, wherein each heterocyclic group has from 4-10 atoms in its ring system, and with the proviso that the ring of said group does not contain two adjacent O atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other illustrative examples of 4-10 membered heterocyclic are derived from, but not limited to, the following:

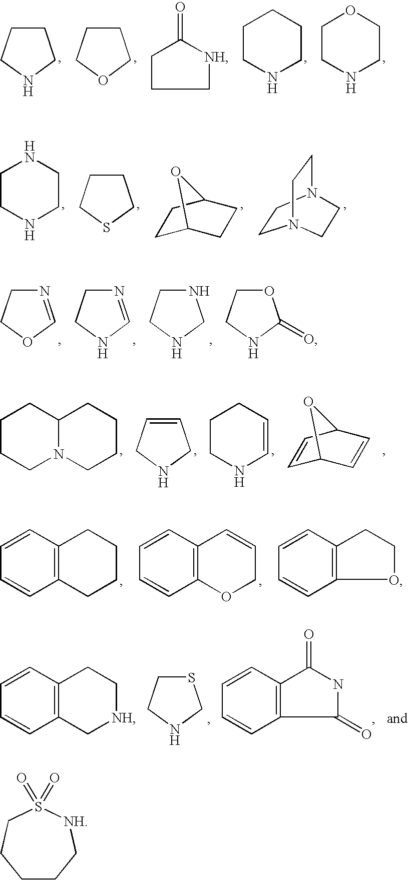

Unless defined otherwise, “alkyl,” “alkylene,” “alkenyl,” “alkynyl,” “aryl,” “cycloalkyl,” or “heterocyclyl” are each optionally and independently substituted by 1-3 substituents selected from alkylamine, amino, aryl, cycloalkyl, heterocyclyl, C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms, carboxyl, cyano, halo, hydroxy, nitro, —C(O)OH, —C(O)₂—(C₁-C₆ alkyl), —C(O)₂—(C₃-C₈ cycloalkyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkyl), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkylene)cycloalkyl, wherein each of these optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents, e.g., CF₃.

The term “immunomodulator” refers to natural or synthetic products capable of modifying the normal or aberrant immune system through stimulation or suppression.

The term “preventing” refers to the ability of a compound or composition of the invention to prevent a disease identified herein in patients diagnosed as having the disease or who are at risk of developing such disease. The term also encompasses preventing further progression of the disease in patients who are already suffering from or have symptoms of such disease.

The term “patient” or “subject” means an animal (e.g., cow, horse, sheep, pig, chicken, turkey, quail, cat, dog, mouse, rat, rabbit, guinea pig, etc.) or a mammal, including chimeric and transgenic animals and mammals. In the treatment or prevention of HCV infection, the term “patient” or “subject” preferably means a monkey or a human, most preferably a human. In a specific embodiment the patient or subject is infected by or exposed to the hepatitis C virus. In certain embodiments, the patient is a human infant (age 0-2), child (age 2-17), adolescent (age 12-17), adult (age 18 and up) or geriatric (age 70 and up) patient. In addition, the patient includes immunocompromised patients such as HIV positive patients, cancer patients, patients undergoing immunotherapy or chemotherapy. In a particular embodiment, the patient is a healthy individual, i.e., not displaying symptoms of other viral infections.

The term a “therapeutically effective amount” refers to an amount of the compound of the invention sufficient to provide a benefit in the treatment or prevention of viral disease, to delay or minimize symptoms associated with viral infection or viral-induced disease, or to cure or ameliorate the disease or infection or cause thereof. In particular, a therapeutically effective amount means an amount sufficient to provide a therapeutic benefit in vivo. Used in connection with an amount of a compound of the invention, the term preferably encompasses a non-toxic amount that improves overall therapy, reduces or avoids symptoms or causes of disease, or enhances the therapeutic efficacy of or synergies with another therapeutic agent.

The term a “prophylactically effective amount” refers to an amount of a compound of the invention or other active ingredient sufficient to result in the prevention of infection, recurrence or spread of viral infection. A prophylactically effective amount may refer to an amount sufficient to prevent initial infection or the recurrence or spread of the infection or a disease associated with the infection. Used in connection with an amount of a compound of the invention, the term preferably encompasses a non-toxic amount that improves overall prophylaxis or enhances the prophylactic efficacy of or synergies with another prophylactic or therapeutic agent.

The term “in combination” refers to the use of more than one prophylactic and/or therapeutic agents simultaneously or sequentially and in a manner that their respective effects are additive or synergistic.

The term “treating” refers to:

(i) preventing a disease, disorder, or condition from occurring in an animal that may be predisposed to the disease, disorder and/or condition, but has not yet been diagnosed as having it;

(ii) inhibiting the disease, disorder, or condition, i.e., arresting its development; and

(iii) relieving the disease, disorder, or condition, i.e., causing regression of the disease, disorder, and/or condition.

The terms “R” and “S” indicate the specific stereochemical configuration of a substituent at an asymmetric carbon atom in a chemical structure as drawn.

The term “rac” indicates that a compound is a racemate, which is defined as an equimolar mixture of a pair of enantiomers. A “rac” compound does not exhibit optical activity. The chemical name or formula of a racemate is distinguished from those of the enantiomers by the prefix (+)- or rac- (or racem-) or by the symbols RS and SR.

The terms “endo” and “exo” are descriptors of the relative orientation of substituents attached to non-bridgehead atoms in a bicyclo[x.y.z]alkane (x≧y>z>0).

The terms “syn” and “anti” are descriptors of the relative orientation of substituents attached to bridgehead atoms in a bicyclo[x.y.z]alkane (x≧y>z>0).

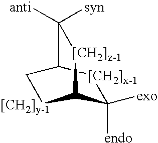

The term “exo” is given to a substituent (e.g., Br attached to C-2 in the example below) that is orientated towards the highest numbered bridge (z bridge, e.g., C-7 in example below); if the substituent is orientated away from the highest numbered bridge it is given the description “endo”.

The term “syn” is given to a substituent attached to the highest numbered bridge (z bridge, e.g., F attached to C-7 in the example below) and is orientated towards the lowest numbered bridge (x bridge, e.g., C-2 and C-3 in example below); if the substituent is orientated away from the lowest numbered bridge it is given the description “anti.”

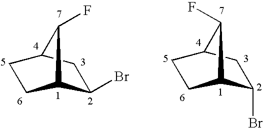

2-exo-bromo-7-syn-fluoro-bicyclo[2.2.1]heptane 2-endo-bromo-7-anti-fluoro-bicyclo[2.2.1]heptane

The terms “cis” and “trans” are descriptors which show the relationship between two ligands attached to separate atoms that are connected by a double bond or are contained in a ring. The two ligands are said to be located cis to each other if they lie on the same side of a plane. If they are on opposite sides, their relative position is described as trans. The appropriate reference plane of a double bond is perpendicular to that of the relevant σ-bonds and passes through the double bond. For a ring it is the mean plane of the ring(s).

The compounds of the invention may exhibit the phenomenon of tautomerism. While Formula I cannot expressly depict all possible tautomeric forms, it is to be understood that Formula I is intended to represent any tautomeric form of the depicted compound and is not to be limited merely to a specific compound form depicted by the formula drawings. For illustration, and in no way limiting the range of tautomers, the compounds of Formula I may exist as the following:

When X═N:

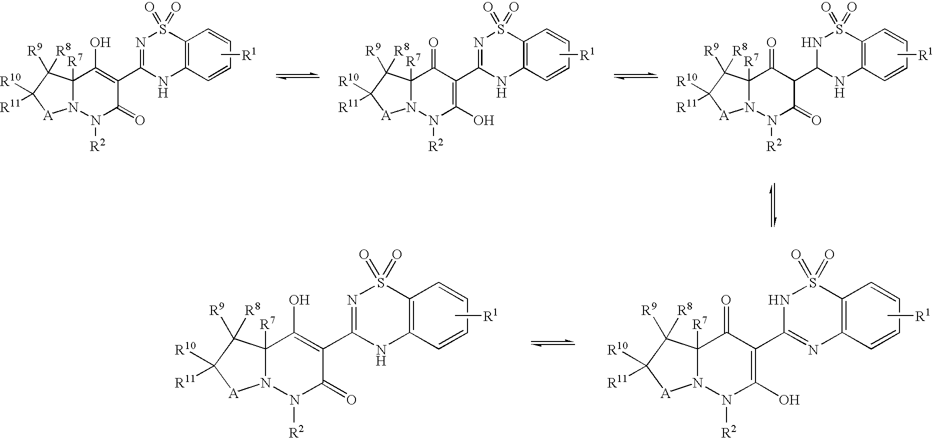

When X═CR³:

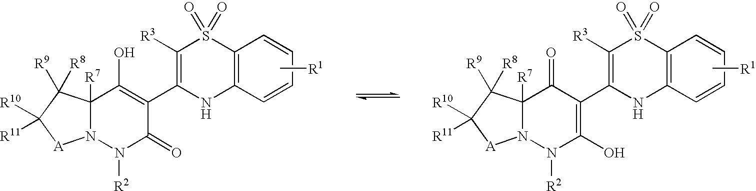

The compounds of Formula I wherein Y is selected from

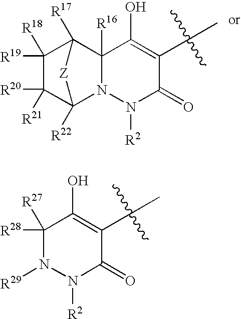

may also exist as tautomers analogous to those depicted above.

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% free of other enantiomers or diastereomers of the compounds, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, the Formula I is intended to cover solvated as well as unsolvated forms of the identified structures. For example, Formula I includes compounds of the indicated structure in both hydrated and non-hydrated forms. Other examples of solvates include the structures in combination with isopropanol, ethanol, methanol, DMSO, ethyl acetate, acetic acid, or ethanolamine.

In addition to compounds of Formula I, the invention includes pharmaceutically acceptable prodrugs, pharmaceutically active metabolites, and pharmaceutically acceptable salts of such compounds and metabolites.

“A pharmaceutically acceptable prodrug” is a compound that may be converted under physiological conditions or by solvolysis to the specified compound or to a pharmaceutically acceptable salt of such compound prior to exhibiting its pharmacological effect (s). Typically, the prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the compounds of Formula I using methods known in the art, such as those described by Burger's Medicinal Chemistry and Drug Chemistry, 1, 172-178, 949-982 (1995). See also Bertolini et al., J. Med. Chem., 40, 2011-2016 (1997); Shan, et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

“A pharmaceutically active metabolite” is intended to mean a pharmacologically active product produced through metabolism in the body of a specified compound or salt thereof. After entry into the body, most drugs are substrates for chemical reactions that may change their physical properties and biologic effects. These metabolic conversions, which usually affect the polarity of the Formula I compounds, alter the way in which drugs are distributed in and excreted from the body. However, in some cases, metabolism of a drug is required for therapeutic effect. For example, anticancer drugs of the anti-metabolite class must be converted to their active forms after they have been transported into a cancer cell.

Since most drugs undergo metabolic transformation of some kind, the biochemical reactions that play a role in drug metabolism may be numerous and diverse. The main site of drug metabolism is the liver, although other tissues may also participate.

A feature characteristic of many of these transformations is that the metabolic products, or “metabolites,” are more polar than the parent drugs, although a polar drug does sometime yield a less polar product. Substances with high lipid/water partition coefficients, which pass easily across membranes, also diffuse back readily from tubular urine through the renal tubular cells into the plasma. Thus, such substances tend to have a low renal clearance and a long persistence in the body. If a drug is metabolized to a more polar compound, one with a lower partition coefficient, its tubular reabsorption will be greatly reduced. Moreover, the specific secretory mechanisms for anions and cations in the proximal renal tubules and in the parenchymal liver cells operate upon highly polar substances.

As a specific example, phenacetin (acetophenetidin) and acetanilide are both mild analgesic and antipyretic agents, but are transformed within the body to a more polar and more effective metabolite, p-hydroxyacetanilid (acetaminophen), which is widely used today. When a dose of acetanilide is given to a person, the successive metabolites peak and decay in the plasma sequentially. During the first hour, acetanilide is the principal plasma component. In the second hour, as the acetanilide level falls, the metabolite acetaminophen concentration reaches a peak. Finally, after a few hours, the principal plasma component is a further metabolite that is inert and can be excreted from the body. Thus, the plasma concentrations of one or more metabolites, as well as the drug itself, can be pharmacologically important.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an α-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

### Methods of Treatment and Prevention of Hepatitis C Viral Infections

The present invention provides methods for treating or preventing a hepatitis C virus infection in a patient in need thereof.

The present invention further provides methods for introducing a therapeutically effective amount of the Formula I compound or combination of such compounds into the blood stream of a patient in the treatment and/or prevention of hepatitis C viral infections.

The magnitude of a prophylactic or therapeutic dose of a Formula I compound of the invention or a pharmaceutically acceptable salt, solvate, or hydrate, thereof in the acute or chronic treatment or prevention of an infection will vary, however, with the nature and severity of the infection, and the route by which the active ingredient is administered. The dose, and in some cases the dose frequency, will also vary according to the infection to be treated, the age, body weight, and response of the individual patient. Suitable dosing regimens can be readily selected by those skilled in the art with due consideration of such factors.

The methods of the present invention are particularly well suited for human patients. In particular, the methods and doses of the present invention can be useful for immunocompromised patients including, but not limited to cancer patients, HIV infected patients, and patients with an immunodegenerative disease. Furthermore, the methods can be useful for immunocompromised patients currently in a state of remission. The methods and doses of the present invention are also useful for patients undergoing other antiviral treatments. The prevention methods of the present invention are particularly useful for patients at risk of viral infection. These patients include, but are not limited to health care workers, e.g., doctors, nurses, hospice care givers; military personnel; teachers; childcare workers; patients traveling to, or living in, foreign locales, in particular third world locales including social aid workers, missionaries, and foreign diplomats. Finally, the methods and compositions include the treatment of refractory patients or patients resistant to treatment such as resistance to reverse transcriptase inhibitors, protease inhibitors, etc.

### Doses

Toxicity and efficacy of the compounds of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage of the compounds for use in humans. The dosage of such compounds lie preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture; alternatively, the dose of the Formula I compound may be formulated in animal models to achieve a circulating plasma concentration range of the compound that corresponds to the concentration required to achieve a fixed magnitude of response. Such information can be used to more accurately determine useful doses in humans. Levels in plasma may be measured, for example, by high performance liquid chromatography.

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which cells that are responsive to the effects of the Formula I compounds are exposed to the ligand and the magnitude of response is measured by an appropriate technique. The assessment of the Formula I compound is then evaluated with respect to the Formula I compound potency and the degree of conversion of the Formula I compound prodrug. Compounds for use in methods of the invention can be tested in suitable animal model systems prior to testing in humans, including but not limited to in rats, mice, chicken, cows, monkeys, rabbits, hamsters, etc. The compounds can then be used in the appropriate clinical trials.

The magnitude of a prophylactic or therapeutic dose of a prodrug of a Formula I compound of the invention or a pharmaceutically acceptable salt, solvate, or hydrate thereof in the acute or chronic treatment or prevention of an infection or condition will vary with the nature and severity of the infection, and the route by which the active ingredient is administered. The dose, and perhaps the dose frequency, will also vary according to the infection to be treated, the age, body weight, and response of the individual patient. Suitable dosing regimens can be readily selected by those skilled in the art with due consideration of such factors. In one embodiment, the dose administered depends upon the specific compound to be used, and the weight and condition of the patient. Also, the dose may differ for various particular Formula I compounds; suitable doses can be predicted on the basis of the aforementioned in vitro measurements and on the basis of animal studies, such that smaller doses will be suitable for those Formula I compounds that show effectiveness at lower concentrations than other Formula I compounds when measured in the systems described or referenced herein. In general, the dose per day is in the range of from about 0.001 to 100 mg/kg, preferably about 1 to 25 mg/kg, more preferably about 5 to 15 mg/kg. For treatment of humans infected by hepatitis C viruses, about 0.1 mg to about 15 g per day is administered in about one to four divisions a day, preferably 100 mg to 12 g per day, more preferably from 100 mg to 8000 mg per day.

Additionally, the recommended daily dose ran can be administered in cycles as single agents or in combination with other therapeutic agents. In one embodiment, the daily dose is administered in a single dose or in equally divided doses. In a related embodiment, the recommended daily dose can be administered once time per week, two times per week, three times per week, four times per week or five times per week.

In one embodiment, the compounds of the invention are administered to provide systemic distribution of the compound within the patient. In a related embodiment, the compounds of the invention are administered to produce a systemic effect in the body.

In another embodiment the compounds of the invention are administered via oral, mucosal (including sublingual, buccal, rectal, nasal, or vaginal), parenteral (including subcutaneous, intramuscular, bolus injection, intraarterial, or intravenous), transdermal, or topical administration. In a specific embodiment the compounds of the invention are administered via mucosal (including sublingual, buccal, rectal, nasal, or vaginal), parenteral (including subcutaneous, intramuscular, bolus injection, intraarterial, or intravenous), transdermal, or topical administration. In a further specific embodiment, the compounds of the invention are administered via oral administration. In a further specific embodiment, the compounds of the invention are not administered via oral administration.

Different therapeutically effective amounts may be applicable for different infections, as will be readily known by those of ordinary skill in the art. Similarly, amounts sufficient to treat or prevent such infections, but insufficient to cause, or sufficient to reduce, adverse effects associated with conventional therapies are also encompassed by the above described dosage amounts and dose frequency schedules.

### Combination Therapy

Specific methods of the invention further comprise the administration of an additional therapeutic agent (i.e., a therapeutic agent other than a compound of the invention). In certain embodiments of the present invention, the compounds of the invention can be used in combination with at least one other therapeutic agent. Therapeutic agents include, but are not limited to antibiotics, antiemetic agents, antidepressants, and antifungal agents, anti-inflammatory agents, antiviral agents, anticancer agents, immunomodulatory agents, α-interferons, β-interferons, ribavirin, alkylating agents, hormones, cytokines, or toll receptor-like modulators. In one embodiment the invention encompasses the administration of an additional therapeutic agent that is HCV specific or demonstrates anti-HCV activity.

The Formula I compounds of the invention can be administered or formulated in combination with antibiotics. For example, they can be formulated with a macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

The Formula I compounds of the invention can also be administered or formulated in combination with an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine, tropisetron, and mixtures thereof.

The Formula I compounds of the invention can be administered or formulated in combination with an antidepressant. Suitable antidepressants include, but are not limited to, binedaline, caroxazone, citalopram, dimethazan, fencamine, indalpine, indeloxazine hydrocholoride, nefopam, nomifensine, oxitriptan, oxypertine, paroxetine, sertraline, thiazesim, trazodone, benmoxine, iproclozide, iproniazid, isocarboxazid, nialamide, octamoxin, phenelzine, cotinine, rolicyprine, rolipram, maprotiline, metralindole, mianserin, mirtazepine, adinazolam, amitriptyline, amitriptylinoxide, amoxapine, butriptyline, clomipramine, demexiptiline, desipramine, dibenzepin, dimetacrine, dothiepin, doxepin, fluacizine, imipramine, imipramine N-oxide, iprindole, lofepramine, melitracen, metapramine, nortriptyline, noxiptilin, opipramol, pizotyline, propizepine, protriptyline, quinupramine, tianeptine, trimipramine, adrafinil, benactyzine, bupropion, butacetin, dioxadrol, duloxetine, etoperidone, febarbamate, femoxetine, fenpentadiol, fluoxetine, fluvoxamine, hematoporphyrin, hypericin, levophacetoperane, medifoxamine, milnacipran, minaprine, moclobemide, nefazodone, oxaflozane, piberaline, prolintane, pyrisuccideanol, ritanserin, roxindole, rubidium chloride, sulpiride, tandospirone, thozalinone, tofenacin, toloxatone, tranylcypromine, L-tryptophan, venlafaxine, viloxazine, and zimeldine.

The Formula I compound s of the invention can be administered or formulated in combination with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofulvin.

The Formula I compounds of the invention can be administered or formulated in combination with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; steroids including, but not limited to, alclometasone diproprionate, amcinonide, beclomethasone dipropionate, betametasone, betamethasone benzoate, betamethasone diproprionate, betamethasone sodium phosphate, betamethasone valerate, clobetasol proprionate, clocortolone pivalate, hydrocortisone, hydrocortisone derivatives, desonide, desoximatasone, dexamethasone, flunisolide, flucoxinolide, flurandrenolide, halcinocide, medrysone, methylprednisolone, methprednisolone acetate, methylprednisolone sodium succinate, mometasone furoate, paramethasone acetate, prednisolone, prednisolone acetate, prednisolone sodium phosphate, prednisolone tebuatate, prednisone, triamcinolone, triamcinolone acetonide, triamcinolone diacetate, and triamcinolone hexacetonide; and other anti-inflammatory agents including, but not limited to, methotrexate, colchicine, allopurinol, probenecid, sulfinpyrazone and benzbromarone.

The Formula I compounds of the invention can be administered or formulated in combination with another antiviral agent. Useful antiviral agents include, but are not limited to, protease inhibitors, nucleoside reverse transcriptase inhibitors, non-nucleoside reverse transcriptase inhibitors and nucleoside analogs. The antiviral agents include but are not limited to zidovudine, acyclovir, gangcyclovir, vidarabine, idoxuridine, trifluridine, levovirin, viramidine and ribavirin, as well as foscarnet, amantadine, rimantadine, saquinavir, indinavir, amprenavir, lopinavir, ritonavir, the α-interferons; β-interferons; adefovir, clevadine, entecavir, pleconaril.

The Formula I compound of the invention can be administered or formulated in combination with an immunomodulatory agent. Immunomodulatory agents include, but are not limited to, methothrexate, leflunomide, cyclophosphamide, cyclosporine A, mycophenolate mofetil, rapamycin (sirolimus), mizoribine, deoxyspergualin, brequinar, malononitriloamindes (e.g., leflunamide), T cell receptor modulators, and cytokine receptor modulators, peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. Examples of T cell receptor modulators include, but are not limited to, anti-T cell receptor antibodies (e.g., anti-CD4 antibodies (e.g., cM-T412 (Boehringer), IDEC-CE9.1® (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131 (IDEC)), anti-CD52 antibodies (e.g., CAMPATH 1H (Ilex)), anti-CD2 antibodies, anti-CD11a antibodies (e.g., Xanelim (Genentech)), anti-B7 antibodies (e.g., IDEC-114 (IDEC)), CTLA4-immunoglobulin, and toll receptor-like (TLR) modulators. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e.g., anti-IFN antibodies, anti-TNF-α antibodies, anti-IL-1β antibodies, anti-IL-6 antibodies, anti-IL-8 antibodies (e.g., ABX-IL-8 (Abgenix)), and anti-IL-12 antibodies).

The Formula I compounds of the invention can be administered or formulated in combination with an agent which inhibits viral enzymes, including but not limited to inhibitors of HCV protease, such as BILN 2061, SCH-503034, ITMN-191 or VX-950; and inhibitors of NS5B polymerase such as NM107 (and its prodrug NM283), R1626, R7078, BILN1941, GSK625433, GILD9128 or HCV-796.

The Formula I compounds of the invention can be administered or formulated in combination with an agent which inhibits HCV polymerase such as those described in Wu, Curr Drug Targets Infect Disord. 2003; 3(3):207-19 or in combination with compounds that inhibit the helicase function of the virus such as those described in Bretner M, et al Nucleosides Nucleotides Nucleic Acids. 2003; 22(5-8):1531, or with inhibitors of other HCV specific targets such as those described in Zhang X., IDrugs, 5(2), 154-8 (2002).

The Formula I compounds of the invention can be administered or formulated in combination with an agent which inhibits viral replication.

The Formula I compounds of the invention can be administered or formulated in combination with cytokines. Examples of cytokines include, but are not limited to, interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), platelet derived growth factor (PDGF), erythropoietin (Epo), epidermal growth factor (EGF), fibroblast growth factor (FGF), granulocyte macrophage stimulating factor (GM-CSF), granulocyte colony stimulating factor (G-CSF), macrophage colony stimulating factor (M-CSF), prolactin, and interferon (IFN), e.g., IFN-α, and IFN-γ).

The Formula I compounds of the invention can be administered or formulated in combination with hormones. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins.

The Formula I compounds of the invention can be administered or formulated in combination with β-interferons which include, but are not limited to, interferon β-1a, interferon β-1b.

The Formula I compounds of the invention can be administered or formulated in combination with α-interferons which include, but are not limited to, interferon α-1, interferon α-2a (roferon), interferon α-2b, intron, Peg-Intron, Pegasys, consensus interferon (infergen) and albuferon.

The Formula I compounds of the invention can be administered or formulated in combination with an absorption enhancer, particularly those which target the lymphatic system, including, but not limited to sodium glycocholate; sodium caprate; N-lauryl-β-D-maltopyranoside; EDTA; mixed micelle; and those reported in Muranishi Crit. Rev. Ther. Drug Carrier Syst., 7-1-33, which is hereby incorporated by reference in its entirety. Other known absorption enhancers can also be used. Thus, the invention also encompasses a pharmaceutical composition comprising one or more Formula I compounds of the invention and one or more absorption enhancers.

The Formula I compounds of the invention can be administered or formulated in combination with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The compounds of the invention and the other therapeutics agent can act additively or, more preferably, synergistically. In one embodiment, a composition comprising a compound of the invention is administered concurrently with the administration of another therapeutic agent, which can be part of the same composition or in a different composition from that comprising the compounds of the invention. In another embodiment, a compound of the invention is administered prior to or subsequent to administration of another therapeutic agent. In a separate embodiment, a compound of the invention is administered to a patient who has not previously undergone or is not currently undergoing treatment with another therapeutic agent, particularly an antiviral agent.

In one embodiment, the methods of the invention comprise the administration of one or more Formula I compounds of the invention without an additional therapeutic agent.

### Pharmaceutical Compositions and Dosage Forms

Pharmaceutical compositions and single unit dosage forms comprising a Formula I compound of the invention, or a pharmaceutically acceptable salt, or hydrate thereof, are also encompassed by the invention. Individual dosage forms of the invention may be suitable for oral, mucosal (including sublingual, buccal, rectal, nasal, or vaginal), parenteral (including subcutaneous, intramuscular, bolus injection, intraarterial, or intravenous), transdermal, or topical administration. Pharmaceutical compositions and dosage forms of the invention typically also comprise one or more pharmaceutically acceptable excipients. Sterile dosage forms are also contemplated.

In an alternative embodiment, pharmaceutical composition encompassed by this embodiment includes a Formula I compound of the invention, or a pharmaceutically acceptable salt, or hydrate thereof, and at least one additional therapeutic agent. Examples of additional therapeutic agents include, but are not limited to, those listed above.

The composition, shape, and type of dosage forms of the invention will typically vary depending on their use. For example, a dosage form used in the acute treatment of a disease or a related disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease or disorder. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990). Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

Typical pharmaceutical compositions and dosage forms comprise one or more carriers, excipients or diluents. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment, and use of formulations.

Anhydrous pharmaceutical compositions and dosage forms of the invention can be prepared using anhydrous or low moisture containing ingredients and low moisture or low humidity conditions.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise Formula I compounds of the invention, or a pharmaceutically acceptable salt or hydrate thereof comprise 0.1 mg to 1500 mg per unit to provide doses of about 0.01 to 200 mg/kg per day.

### Oral Dosage Forms

Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredient(s) in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Examples of excipients that can be used in oral dosage forms of the invention include, but are not limited to, binders, fillers, disintegrants, and lubricants. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Suitable forms of microcrystalline cellulose include, but are not limited to, the materials sold as AVICEL-PH-101, AVICEL-PH-103 AVICEL RC-581, AVICEL-PH-105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, specifically from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

### Delayed Release Dosage Forms

Active ingredients of the invention can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719, 5,674,533, 5,059,595, 5,591,767, 5,120,548, 5,073,543, 5,639,476, 5,354,556, and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

### Parenteral Dosage Forms

Parenteral dosage forms can be administered to patients by various routes including, but not limited to, subcutaneous, intravenous (including bolus injection), intramuscular, and intraarterial. Because their administration typically bypasses patients' natural defenses against contaminants, parenteral dosage forms are preferably sterile or capable of being sterilized prior to administration to a patient. Examples of parenteral dosage forms include, but are not limited to, solutions ready for injection, dry and/or lyophylized products ready to be dissolved or suspended in a pharmaceutically acceptable vehicle for injection (reconstitutable powders), suspensions ready for injection, and emulsions.

Suitable vehicles that can be used to provide parenteral dosage forms of the invention are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Compounds that increase the solubility of one or more of the active ingredients disclosed herein can also be incorporated into the parenteral dosage forms of the invention.

### Transdermal Dosage Forms

Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

The pH of a pharmaceutical composition or dosage form, or of the tissue to which the pharmaceutical composition or dosage form is applied, may also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Topical Dosage Forms

Topical dosage forms of the invention include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985).

Suitable excipients (e.g., carriers and diluents) and other materials that can be used to provide transdermal and topical dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane-1,3-diol, isopropyl myristate, isopropyl palmitate, mineral oil, and mixtures thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

### Mucosal Dosage Forms

Mucosal dosage forms of the invention include, but are not limited to, ophthalmic solutions, sprays and aerosols, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. In one embodiment, the aerosol comprises a carrier. In another embodiment, the aerosol is carrier free.

The Formula I compounds of the invention may also be administered directly to the lung by inhalation. For administration by inhalation, a Formula I compound can be conveniently delivered to the lung by a number of different devices. For example, a Metered Dose Inhaler (“MDI”) which utilizes canisters that contain a suitable low boiling propellant, e.g., dichlorodifluoromethane, trichlorofluoromethane, dichlorotetrafluoroethane, carbon dioxide or other suitable gas can be used to deliver a Formula I compound directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device can be used to administer a Formula I compound to the lung (see, e.g., Raleigh et al., Proc. Amer. Assoc. Cancer Research Annual Meeting, 1999, 40, 397, which is herein incorporated by reference). DPI devices typically use a mechanism such as a burst of gas to create a cloud of dry powder inside a container, which can then be inhaled by the patient. DPI devices are also well known in the art and can be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator can be formulated containing a powder mix of the compound and a suitable powder base such as lactose or starch for these systems.

Another type of device that can be used to deliver a Formula I compound to the lung is a liquid spray device supplied, for example, by Aradigm Corporation. Liquid spray systems use extremely small nozzle holes to aerosolize liquid drug formulations that can then be directly inhaled into the lung.

In one embodiment, a nebulizer device is used to deliver a Formula I compound to the lung. Nebulizers create aerosols from liquid drug formulations by using, for example, ultrasonic energy to form fine particles that can be readily inhaled (See e.g., Verschoyle et al., British J. Cancer, 1999, 80, Suppl 2, 96, which is herein incorporated by reference). Examples of nebulizers include devices supplied by Sheffield/Systemic Pulmonary Delivery Ltd. (See, Armer et al., U.S. Pat. No. 5,954,047; van der Linden et al., U.S. Pat. No. 5,950,619; van der Linden et al., U.S. Pat. No. 5,970,974, which are herein incorporated by reference), Aventis and Batelle Pulmonary Therapeutics.

In one embodiment, an electrohydrodynamic (“EHD”) aerosol device is used to deliver Formula I compounds to the lung. EHD aerosol devices use electrical energy to aerosolize liquid drug solutions or suspensions (see, e.g., Noakes et al., U.S. Pat. No. 4,765,539; Coffee, U.S. Pat. No. 4,962,885; Coffee, PCT Application, WO 94/12285; Coffee, PCT Application, WO 94/14543; Coffee, PCT Application, WO 95/26234, Coffee, PCT Application, WO 95/26235, Coffee, PCT Application, WO 95/32807, which are herein incorporated by reference). The electrochemical properties of the Formula I compounds formulation may be important parameters to optimize when delivering this drug to the lung with an EHD aerosol device and such optimization is routinely performed by one of skill in the art. EHD aerosol devices may more efficiently delivery drugs to the lung than existing pulmonary delivery technologies. Other methods of intra-pulmonary delivery of Formula I compounds will be known to the skilled artisan and are within the scope of the invention.

Liquid drug formulations suitable for use with nebulizers and liquid spray devices and EHD aerosol devices will typically include a Formula I compound with a pharmaceutically acceptable carrier. Preferably, the pharmaceutically acceptable carrier is a liquid such as alcohol, water, polyethylene glycol or a perfluorocarbon. Optionally, another material may be added to alter the aerosol properties of the solution or suspension of the Formula I compound. Preferably, this material is liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. Nos. 5,112,598; Biesalski, 5,556,611, which are herein incorporated by reference) A Formula I compound can also be formulated in rectal or vaginal compositions such as suppositories or retention enemas, e.g., containing conventional suppository bases such as cocoa butter or other glycerides.

In addition to the formulations described previously, a Formula I compound can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other pharmaceutical delivery systems can be employed. Liposomes and emulsions are well known examples of delivery vehicles that can be used to deliver Formula I compounds. Certain organic solvents such as dimethylsulfoxide can also be employed, although usually at the cost of greater toxicity. A Formula I compound can also be delivered in a controlled release system. In one embodiment, a pump can be used (Sefton, CRC Crit. Ref Biomed Eng., 1987, 14, 201; Buchwald et al., Surgery, 1980, 88, 507; Saudek et al., N. Engl. J. Med., 1989, 321, 574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem., 1983, 23, 61; see also Levy et al., Science, 1985, 228, 190; During et al., Ann. Neurol., 1989, 25, 351; Howard et al., J. Neurosurg., 71, 105 (1989). In yet another embodiment, a controlled-release system can be placed in proximity of the target of the compounds of the invention, e.g., the lung, thus requiring only a fraction of the systemic dose (see, e.g., Goodson, in Medical Applications of Controlled Release, supra, vol. 2, pp. 115 (1984)). Other controlled-release system can be used (see, e.g., Langer, Science, 1990, 249, 1527).

Suitable excipients (e.g., carriers and diluents) and other materials that can be used to provide mucosal dosage forms encompassed by this invention are well known to those skilled in the pharmaceutical arts, and depend on the particular site or method which a given pharmaceutical composition or dosage form will be administered. With that fact in mind, typical excipients include, but are not limited to, water, ethanol, ethylene glycol, propylene glycol, butane-1,3-diol, isopropyl myristate, isopropyl palmitate, mineral oil, and mixtures thereof, which are non-toxic and pharmaceutically acceptable. Examples of such additional ingredients are well known in the art. See, e.g., Remington's Pharmaceutical Sciences, 18th eds., Mack Publishing, Easton Pa. (1990).

The pH of a pharmaceutical composition or dosage form, or of the tissue to which the pharmaceutical composition or dosage form is applied, can also be adjusted to improve delivery of one or more active ingredients. Similarly, the polarity of a solvent carrier, its ionic strength, or tonicity can be adjusted to improve delivery. Compounds such as stearates can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant, and as a delivery-enhancing or penetration-enhancing agent. Different salts, hydrates or solvates of the active ingredients can be used to further adjust the properties of the resulting composition.

### Kits

The invention provides a pharmaceutical pack or kit comprising one or more containers comprising a Formula I compound useful for the treatment or prevention of a Hepatitis C virus infection. In other embodiments, the invention provides a pharmaceutical pack or kit comprising one or more containers comprising a Formula I compound useful for the treatment or prevention of a Hepatitis C virus infection and one or more containers comprising an additional therapeutic agent, including but not limited to those listed above, in particular an antiviral agent, an interferon, an agent which inhibits viral enzymes, or an agent which inhibits viral replication, preferably the additional therapeutic agent is HCV specific or demonstrates anti-HCV activity.

The invention also provides a pharmaceutical pack or kit comprising one or more containers comprising one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

The inventive agents may be prepared using the reaction routes and synthesis schemes as described below, employing the general techniques known in the art using starting materials that are readily available. The synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or generally known in the art will be recognized as having applicability for preparing other compounds of the invention.

### Preparation of Compounds

In the synthetic schemes described below, unless otherwise indicated all temperatures are set forth in degrees Celsius and all parts and percentages are by weight.

Reagents were purchased from commercial suppliers such as Aldrich Chemical Company or Lancaster Synthesis Ltd. and were used without further purification unless otherwise indicated. All solvents were purchased from commercial suppliers such as Aldrich, EMD Chemicals or Fisher and used as received.

The reactions set forth below were done generally under a positive pressure of argon or nitrogen at an ambient temperature (unless otherwise stated) in anhydrous solvents, and the reaction flasks were fitted with rubber septa for the introduction of substrates and reagents via syringe. Glassware was oven dried and/or heat dried.

The reactions were assayed by TLC and/or analyzed by LC-MS and terminated as judged by the consumption of starting material. Analytical thin layer chromatography (TLC) was performed on glass-plates precoated with silica gel 60 F₂₅₄ 0.25 mm plates (EMD Chemicals), and visualized with UV light (254 nm) and/or iodine on silica gel and/or heating with TLC stains such as ethanolic phosphomolybdic acid, ninhydrin solution, potassium permanganate solution or ceric sulfate solution. Preparative thin layer chromatography (prepTLC) was performed on glass-plates precoated with silica gel 60 F₂₅₄ 0.5 mm plates (20×20 cm, from Thomson Instrument Company) and visualized with UV light (254 nm).

Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ and/or MgSO₄ prior to filtration and evaporation of the solvents under reduced pressure on a rotary evaporator and noted as solvents removed in vacuo. Column chromatography was completed under positive pressure using Merck silica gel 60, 230-400 mesh or 50-200 mesh neutral alumina, ISCO flash column chromatography using prepacked RediSep silica gel columns, or Analogix flash column chromatography using prepacked SuperFlash silica gel columns. Hydrogenolysis was done at the pressure indicated in the examples or at ambient pressure.

¹H-NMR spectra and ¹³C-NMR were recorded on a Varian Mercury-VX400 instrument at 400 MHz or 100 MHz respectively. NMR spectra were obtained as CDCl₃ solutions (reported in ppm), using chloroform as the reference standard (7.27 ppm for the proton and 77.00 ppm for carbon), CD₃OD (3.4 and 4.8 ppm for the protons and 49.3 ppm for carbon), DMSO-d₆ (2.49 ppm for proton), or internally tetramethylsilane (0.00 ppm) when appropriate. Other NMR solvents were used as needed. When peak multiplicities are reported, the following abbreviations are used: s (singlet), d (doublet), t (triplet), q (quartet), m (multiplet), br (broadened), bs (broad singlet), dd (doublet of doublets), dt (doublet of triplets). Coupling constants, when given, are reported in Hertz (Hz).

Infrared (IR) spectra were recorded on an ATR FT-IR Spectrometer as neat oils or solids, and when given are reported in wave numbers (cm⁻¹). Mass spectra reported are (+)-ES or APCI (+) LC/MS conducted by the Analytical Chemistry Department of Anadys Pharmaceuticals, Inc. Elemental analyses were conducted by the Atlantic Microlab, Inc. in Norcross, Ga. Melting points (mp) were determined on an open capillary apparatus, and are uncorrected.

The described synthetic pathways and experimental procedures utilize many common chemical abbreviations, 2,2-DMP (2,2-dimethoxypropane), Ac (acetyl), ACN (acetonitrile), Bn (benzyl), BnOH (benzyl alcohol), Boc (tert-butoxycarbonyl), Boc₂O (di-tert-butyl dicarbonate), Bz (benzoyl), CSI (chlorosulfonyl isocyanate), DBU (1,8-diazabicyclo[5,4,0]undec-7-ene), DCC (N,N′-dicyclohexylcarbodiimide), DCE (1,2-dichloroethane), DCM (dichloromethane), DEAD (diethylazodicarboxylate), DIEA (diisopropylethylamine), DMA (N,N-dimethylacetamide), DMAP (4-(N,N-dimethylamino)pyridine), DMF (N,N-dimethylformamide), DMSO (dimethyl sulfoxide), EDC (1-(3-dimethylaminopropyl)-3-ethylcarbodiimide hydrochloride), Et (ethyl), EtOAc (ethyl acetate), EtOH (ethanol), HATU (O-(7-azabenzotriazol-1-yl)-1,1,3,3-tetramethyluronium hexafluorophosphate), HBTU (O-benzotriazol-1-yl-N,N,N′,N′-tetramethyluronium hexafluorophosphate), HF (hydrogen fluoride), HOAc (acetic acid), HOBT (1-hydroxybenzotriazole hydrate), HPLC (high pressure liquid chromatography), IPA (isopropyl alcohol), KHMDS (potassium bis(trimethylsilyl)amide), KN(TMS)₂ (potassium bis(trimethylsilyl)amide), KO^{t}Bu (potassium tert-butoxide), LDA (lithium diisopropylamine), MCPBA (3-chloroperbenzoic acid), Me (methyl), MeCN (acetonitrile), MeOH (methanol), NaCNBH₃ (sodium cyanoborohydride), NaH (sodium hydride), NaN(TMS)₂ (sodium bis(trimethylsilyl)amide), NaOAc (sodium acetate), NaOEt (sodium ethoxide), NMM (N-methylmorpholine), Phe (phenylalanine), PPTS (pyridinium p-toluenesulfonate), PS (polymer supported), Py (pyridine), pyBOP (benzotriazol-1-yloxy)tripyrrolidinophosphonium hexafluorophosphate), TEA (triethylamine), TFA (trifluoroacetic acid), TFAA (trifluoroacetic anhydride), THF (tetrahydrofuran), TLC (thin layer chromatography), Tol (toluoyl), Val (valine), and the like.

Scheme 1 provides a general procedure that can be used to prepare saturated fused [1,2-b]pyridazinone compounds of Formula I.

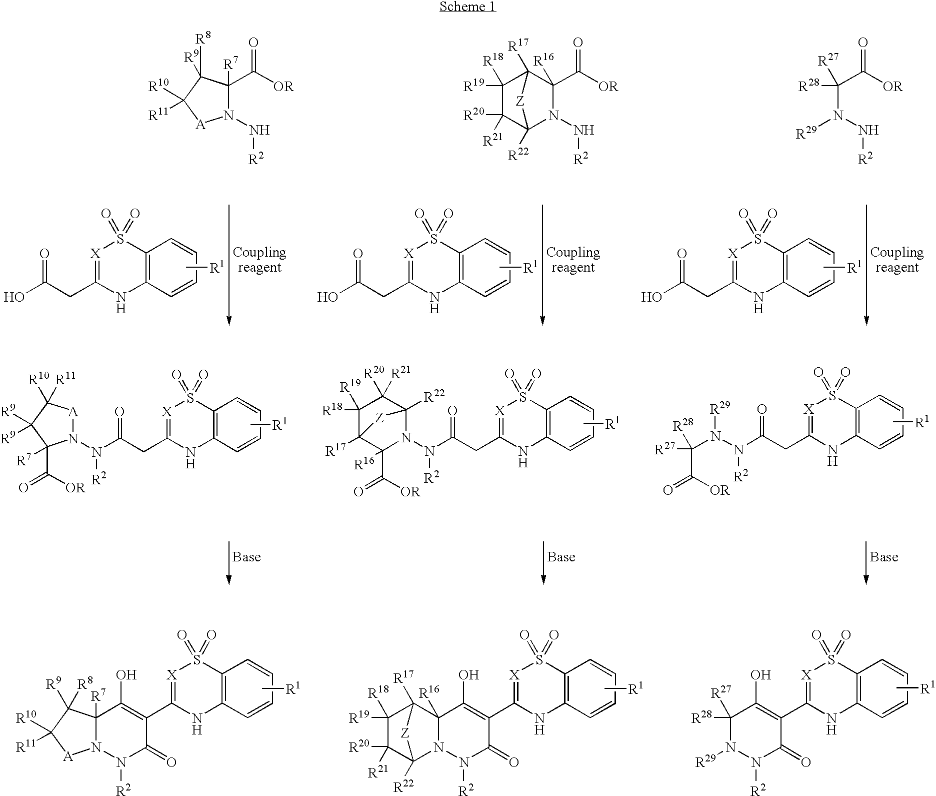

The N-substituted hydrazine intermediates, which can be obtained as described below, can be condensed with a carboxylic acid intermediate using standard peptide coupling conditions used for the formation of amide bonds, such as DCC or EDC, to yield the shown hydrazide intermediates. These intermediates can be cyclized in the presence of a base, such as NaOEt or DBU, to give the desired saturated fused [1,2-b]pyridazin-2-one compounds.

Scheme 2 provides a specific procedure that was used to prepare a saturated fused [1,2-b]pyridazinone compound of Formula I.

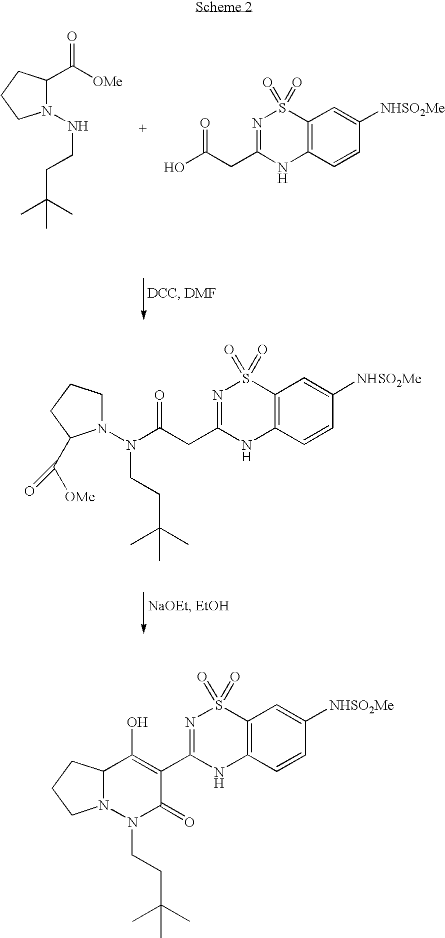

The N-substituted hydrazine intermediate, which can be obtained as described below, can be condensed with a carboxylic acid intermediate using standard peptide coupling conditions used for the formation of amide bonds, such as DCC or EDC, to yield the shown intermediate. The hydrazide can be cyclized in the presence of a base, such as NaOEt or DBU, to give the desired N-{3-[1-(3,3-dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide.

Scheme 3 provides a general procedure that can be used to prepare saturated N-alkylated fused [1,2-b]pyridazinone compounds of Formula I.

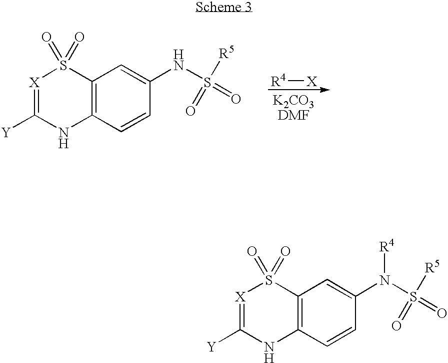

The saturated fused [1,2-b]pyridazinone compounds prepared as described in Schemes 1 and 2, can be N-alkylated using an alkylating agent, such as an alkyl halide, in the presence of a base, e.g., potassium carbonate, to afford the desired saturated N-alkylated fused [1,2-b]pyridazinone compounds.

Scheme 4 provides a general procedure that can be used to prepare the N-substituted hydrazine intermediates.

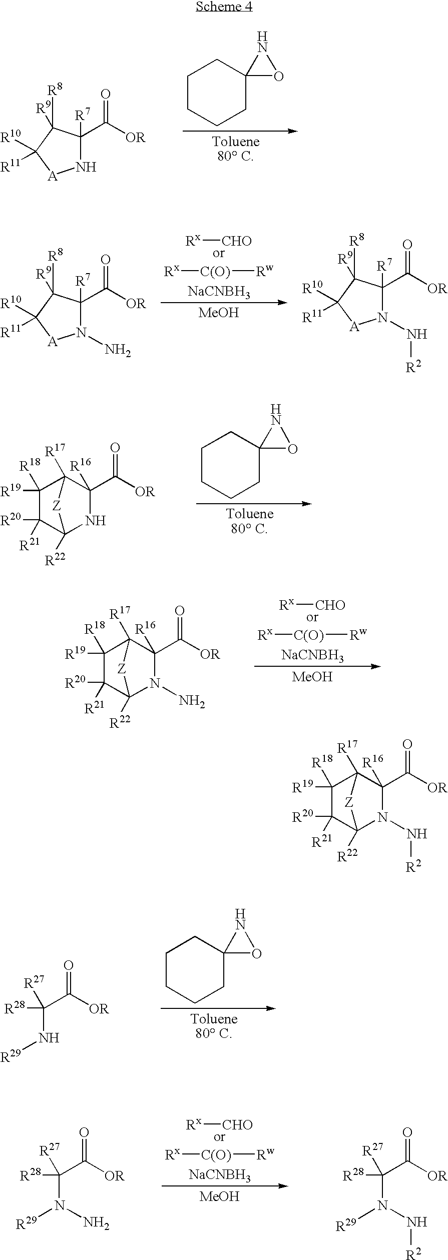

The amino acid esters shown above can be N-aminated using reagents such as 1-oxa-2-aza-spiro[2.5]octane to give the corresponding hydrazine compound. These entities can be N-alkylated by reacting them with aldehydes or ketones, where R^{x} and R^{w} are C₁-C₅ alkyl, C₃-C₈ cycloalkyl, —C₁-C₅ alkylene(C₃-C₈ cycloalkyl), aryl, or heterocyclyl, or R^{w} can combine with R^{x} to form a 3- to 8-membered ring, and a reducing agent, such as sodium cyanoborohydride, to afford the desired N-substituted hydrazine intermediates.

Scheme 5 provides a specific procedure that was used to prepare an N-substituted hydrazine intermediate.

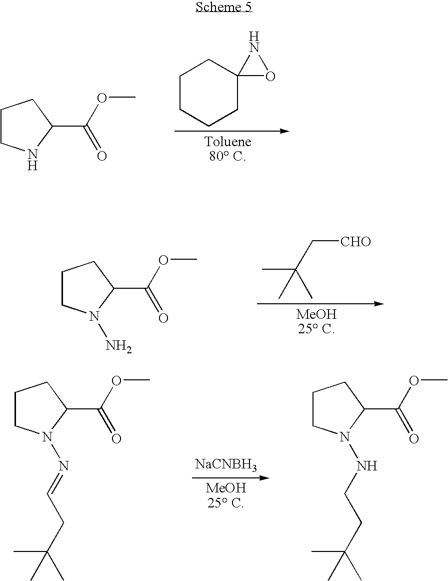

The cyclic amino acid ester (e.g., proline methyl ester) can be N-aminated using reagents such as 1-oxa-2-aza-spiro[2.5]octane to give the corresponding hydrazine compound. Reaction with an aliphatic or aromatic aldehyde gives the imine which can then be reduced with a reducing agent, such as sodium cyanoborohydride, to afford the desired 1-(3,3-dimethyl-butylamino)-pyrrolidine-2-carboxylic acid methyl ester intermediate.

Scheme 6 provides a general procedure that was used to prepare the (7-methanesulfonylamino-1,1-dioxo-1,4-dihydro-1λ⁶-benzo[1,2,4]thiadiazin-3-yl)-acetic acid intermediate.

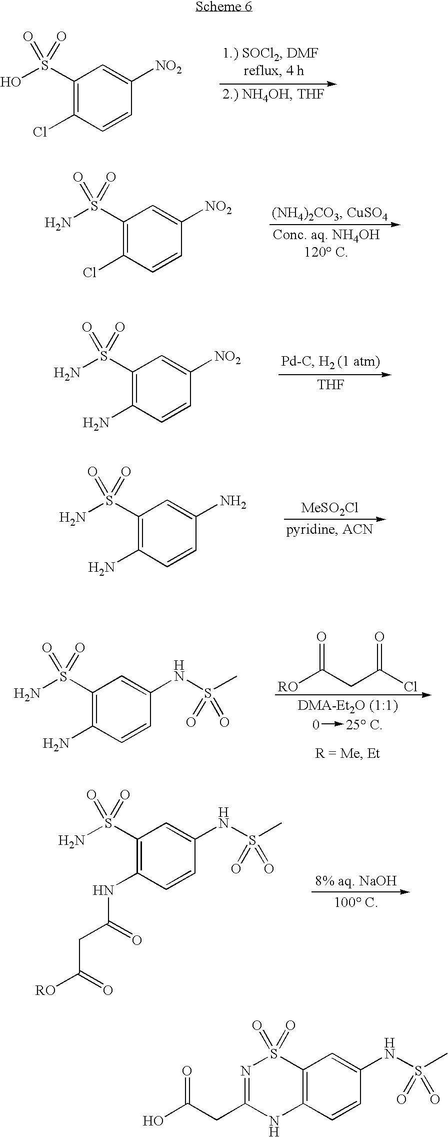

Commercially available 2-chloro-5-nitro-benzenesulfonic acid can be treated with thionyl chloride to give the sulfonylchloride, which can be further treated with ammonia to afford the sulfonamide intermediate. The chloride can be displaced with ammonia by treatment with ammonium hydroxide and ammonium carbonate in the presence of copper(II)sulfate. Reduction of the nitro group under standard hydrogenation conditions affords the aniline intermediate, which can be treated with a sulfonyl chloride, such as methylsulfonyl chloride, to yield the corresponding sulfonamide. Acylation of the 2-amino moiety with malonyl chlorides, e.g., ethyl 3-chloro-3-oxo-propionate, gives the corresponding amide, which can simultaneously be cyclized to the thiadiazine-dioxide and hydrolyzed to the desired acid intermediate.

Scheme 7 provides a general procedure that can be used to prepare the (7-methanesulfonylamino-1,1-dioxo-1,4-dihydro-1λ⁶-benzo[1,4]thiazin-3-yl)-acetic acid ethyl ester intermediate.

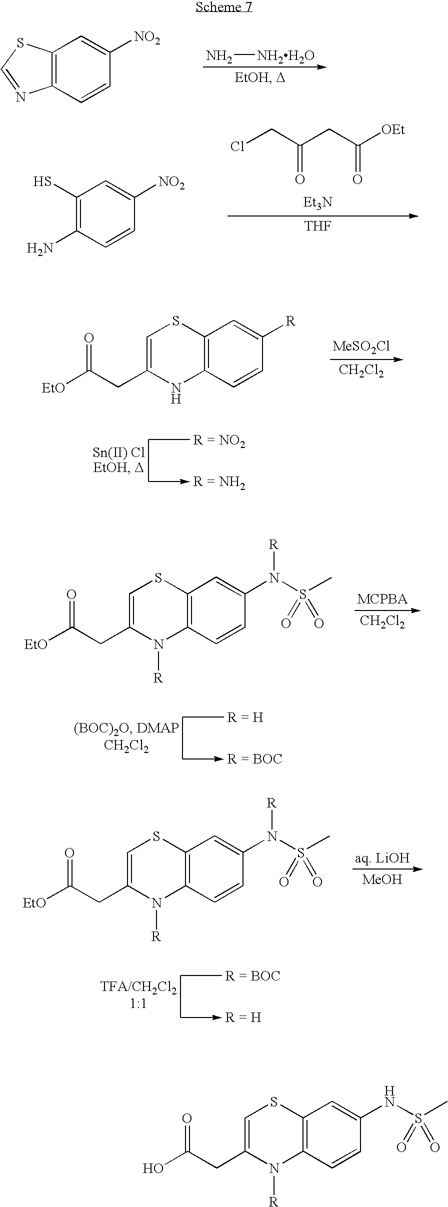

Commercially available 6-nitrobenzothiazole can be treated with hydrazine to obtain the 2-amino-5-nitro-benzenethiol, which can subsequently be reacted with chloroacetoacetate to give the (7-nitro-4H-benzo[1,4]thiazin-3-yl)-acetic acid ethyl ester. Reduction of the nitro group to the amino group can be accomplished by reaction with tin(II)chloride. Subsequent reaction with methansulfonyl chloride can be used to obtain the corresponding sulfonamide. Protection of both nitrogens with a suitable protecting group such as a Boc group can be achieved by using standard methods for protecting amino groups. The sulfide can be oxidized using a suitable oxidizing reagent (e.g., MCPBA) to give the sulfone. Finally, deprotection of the amino groups using trifluoroacetic acid followed by hydrolysis of the ester using standard conditions (e.g., lithium hydroxide) can be used to afford the desired (7-methanesulfonylamino-1,1-dioxo-1,4-dihydro-1λ⁶-benzo[1,4]thiazin-3-yl)-acetic acid intermediate.

Scheme 8 provides a general procedure that can be used to prepare 7-(N-methyl)-substituted-1,1-dioxo-1,4-dihydro-1λ⁶-benzo[1,4]thiazin-3-yl-acetic acid intermediate.

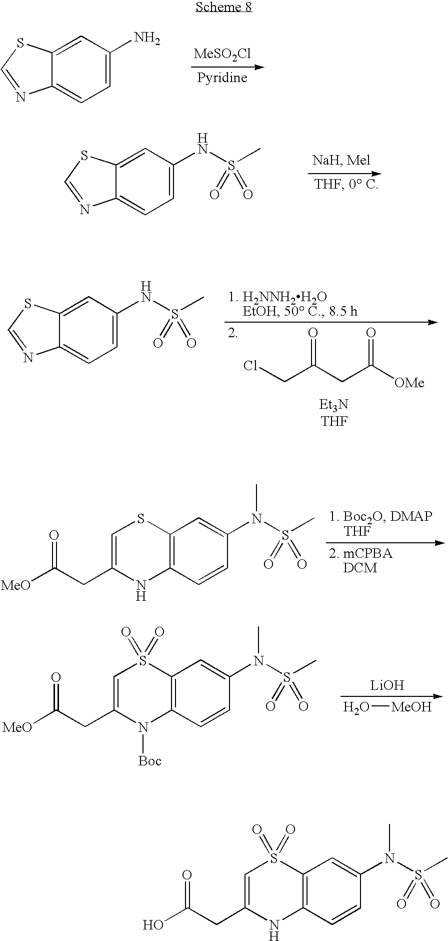

Commercially available 6-aminobenzothiazole can be treated with a sulfonyl chloride, such as methanesulfonyl chloride, to obtain the corresponding sulfonamides. Reaction with methyl iodide, in the presence of a base, gives the corresponding N-methyl sulfonamide. Reaction with hydrazine hydrate and subsequent treatment with methyl chloroacetoacetate affords the corresponding 4H-benzo[1,4]thiazin-3-yl)-acetic acid methyl ester. Protection of the ring nitrogen with a suitable protecting group such as a Boc group can be achieved by using standard methods for protecting amino groups. The sulfide can be oxidized using a suitable oxidizing reagent (e.g., MCPBA) to give the sulfone. Finally, hydrolysis of the ester affords the desired [7-(methanesulfonyl-methyl-amino)-1,1-dioxo-1,4-dihydro-1λ⁶-benzo[1,4]thiazin-3-yl]-acetic acid intermediate.

### EXAMPLE 1

### N-{3-[1-(3,3-Dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide

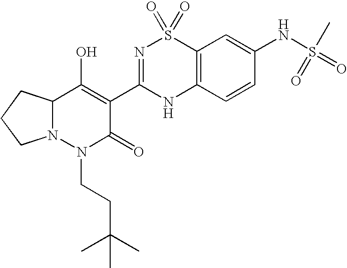

### a) 2-Chloro-5-nitrobenzenesulfonamide

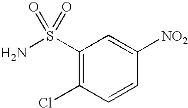

To a solution of thionyl chloride (11 mL) and 2-chloro-5-nitro-benzenesulfonic acid (4.78 g, 20.1 mmol) was added N,N-dimethylformamide (0.92 μL) and the reaction mixture was heated to reflux for 4 h. Upon cooling, the reaction mixture was azeotroped with toluene (2-3×). The sulfonyl chloride was dissolved in a minimal amount of toluene and then added to a mixture of concentrated aqueous ammonium hydroxide solution (25 mL) and tetrahydrofuran (25 mL) at −10° C. After stirring for 2 h the reaction was quenched by adding a 6.0 M aqueous hydrochloric acid solution until pH 4 was reached. The layers were separated and the organic layer was concentrated in vacuo to a slurry. Pentane was added and the product was isolated by vacuum filtration to afford the desired product, 2-chloro-5-nitrobenzenesulfonamide (2.0 g, 8.48 mmol, 42.4% yield) as a solid.

### b) 2-Amino-5-nitrobenzenesulfonamide

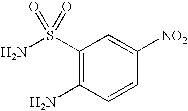

A mixture of 2-chloro-5-nitrobenzenesulfonamide (0.88 g, 3.72 mmol), ammonium carbonate (0.88 g, 9.16 mmol), and copper(II)sulfate (0.175 g, 1.10 mmol) in concentrated aqueous ammonium hydroxide solution (4.4 mL) was heated for 4 h at 120° C. in a pressure reaction vessel. The mixture was allowed to cool to 25° C. and the resulting solid was collected by vacuum filtration, washed with water and dried to afford the desired product, 2-amino-5-nitrobenzenesulfonamide (0.295 g, 1.36 mmol, 36.5% yield) as a tan solid.

### c) 2,5-Diaminobenzenesulfonamide

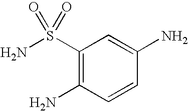

A mixture of 2-amino-5-nitrobenzenesulfonamide (10 g, 46.08 mmol), 10% palladium on charcoal (˜1 g) in tetrahydrofuran (250 mL) was hydrogenated for 26 h at 25° C. under 1 atmosphere of hydrogen gas via balloon. The mixture was then filtered through Celite, washed with tetrahydrofuran, and the solvent removed in vacuo to afford the desired product. The catalyst/Celite mixture was slurried in methanol (400 mL) for 16 h, filtered and the solvent was removed in vacuo to afford a second batch of the desired product, 2,5-diaminobenzenesulfonamide (combined: 7.79 g, 41.65 mmol, 90.4% yield) as a light-brown solid. ¹H NMR (400 MHz, DMSO-d₆) δ: 4.54 (2H, bs), 4.98 (2H, bs), 6.55-6.60 (2H, m), 6.87 (1H, d, J=2.2 Hz), 6.99 (2H, bs). LC-MS (ESI) calcd for C₆H₉N₃O₂S 187.04, found 188.3 [M+H⁺].

### d) 2-Amino-5-methanesulfonylamino-benzenesulfonamide

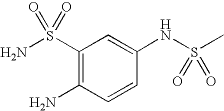

2,5-Diaminobenzenesulfonamide (11.16 g, 59.61 mmol) was dissolved in acetonitrile (300 mL) and pyridine (7.07 g, 89.41 mmol) was added. Methanesulfonyl chloride (7.17 g, 62.59 mmol) was added dropwise over a period of 10 min and the reaction mixture was stirred for 16 h at 25° C. after which time a precipitate had formed. Most of the acetonitrile was removed in vacuo and water (200 mL) was added to afford a clear solution. The product slowly started to precipitate and the mixture was placed in an ice bath for 3 h. The precipitate was collected by vacuum filtration and dried under high vacuum to afford the desired product, 2-amino-5-methanesulfonylamino-benzenesulfonamide (11.1 g, 41.84 mmol, 70.2% yield) as a brown solid. ¹H NMR (400 MHz, CD₃OD) δ: 2.89 (3H, s), 6.82 (1H, d, J=8.5 Hz), 7.20 (1H, dd, J₁=8.5 Hz, J₂=2.5 Hz), 7.58 (1H, d, J=2.5 Hz). LC-MS (ESI) calcd for C₇H₁₁N₃O₄S₂ 265.02, found 266.0 [M+H⁺].

### e) N-(4-Methanesulfonylamino-2-sulfamoyl-phenyl)-malonamic acid ethyl ester

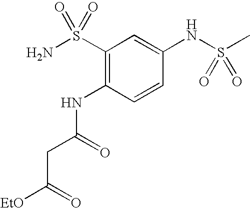

2-Amino-5-methanesulfonylamino-benzenesulfonamide (23.27 g, 87.81 mmol) was dissolved in N,N-dimethylacetamide (100 mL) and diethyl ether (100 mL). Ethyl 3-chloro-3-oxo-propionate (13.88 g, 92.20 mmol) was added and the reaction mixture was stirred at 25° C. for 1 h. The reaction mixture was diluted with ethyl acetate (400 mL) and was extracted with water (400 mL). The aqueous layer was back-extracted with ethyl acetate (2×200 mL). The combined organic layers were dried over sodium sulfate, filtered and most of the solvent was removed in vacuo to a volume of ˜100 mL. To the stirred solution was added hexanes (˜100 mL) upon which a precipitate formed. The precipitate was collected by vacuum filtration, washed with hexanes and dried under high vacuum to afford the analytically pure product, N-(4-methanesulfonylamino-2-sulfamoyl-phenyl)-malonamic acid ethyl ester (31.22 g, 85.53 mmol, 97.4% yield) as a light-brown solid. ¹H NMR (400 MHz, CD₃OD) δ: 1.31 (3H, t, J=7.0 Hz), 3.00 (3H, s), 3.59 (2H, s), 4.25 (2H, q, J=6.9 Hz), 7.42-7.45 (1H, m), 7.86 (1H, m), 7.92 (1H, d, J=8.8 Hz).

### f) (7-Methanesulfonylamino-1,1-dioxo-1,4-dihydro-1λ⁶ benzo[1,2,4]thiadiazin-3-yl)-acetic acid

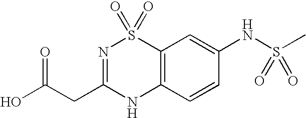

N-(4-Methanesulfonylamino-2-sulfamoyl-phenyl)-malonamic acid ethyl ester (9.55 g, 26.16 mmol) was dissolved in 8% aqueous sodium hydroxide solution (262 mL) and heated at 100° C. for 1.5 h. The reaction mixture was cooled to 0° C. and the solution was acidified by slowly adding 12.0 M aqueous hydrochloric acid solution until pH 1-2 was reached. A precipitate started to form and the suspension was allowed to stir for 30 min at 0° C. The precipitate was collected by vacuum filtration, washed with cold water, and dried under high vacuum to afford (7-methanesulfonylamino-1,1-dioxo-1,4-dihydro-1λ⁶-benzo[1,2,4]thiadiazin-3-yl)-acetic acid (7.20 g, 21.621 mmol, 82.6% yield) as a pinkish solid. ¹H NMR (400 MHz, DMSO-d₆) δ: 3.03 (3H, s), 3.56 (2H, s), 7.33 (1H, d, J=9.1 Hz), 7.52-7.54 (2H, m), 10.09 (1H, s), 12.24 (1H, s), 13.02 (1H, bs). LC-MS (ESI) calcd for C₁₀H₁₁N₃O₆S₂ 333.01, found 334.1 [M+H⁺].

### g) 1-Oxa-2-aza-spiro[2.5]octane

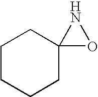

Toluene (250 mL), cyclohexanone (40 mL, 0.4 mol), 2.0 M aqueous ammonium hydroxide solution (50 mL), water (125 mL) and ice (100 g) were combined in a separatory funnel, and the mixture was shaken vigorously. A 1.0 M aqueous sodium hypochlorite solution (100 mL) was added before the two phases were separated and the mixture was shaken again vigorously. The phases were separated, and the organic layer was dried over sodium sulfate and filtered. The toluene solution (kept cold) was used directly in next step, assuming a concentration of 0.28 M based on a literature yield of about 70%. Reference: E. Schmitz et al. J. Prakt. Chem. 1977, 319, 195-200.

### h) 1-(3,3-Dimethyl-butylideneamino)-pyrrolidine-2-carboxylic acid methyl ester

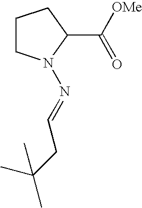

The above 0.28 M solution of 1-oxa-2-aza-spiro[2.5]octane in toluene (79 mL, 22 mmol) was added to DL-proline methyl ester (1.42 g, 11 mmol) and stirred under an inert atmosphere at 80° C. for 16 h. The mixture was cooled to 0° C. and a 10% aqueous sulfuric acid solution (25 mL) was added. The toluene phase was separated and extracted with an ice-cold 10% aqueous sulfuric acid solution (3×10 mL). The combined cold aqueous layers were washed with diethyl ether (2×15 mL), and carefully concentrated in vacuo at 25° C. to remove the remaining cyclohexanone. Crushed ice (70 g) was added and the stirred acidic solution was neutralized by dropwise addition of saturated aqueous sodium bicarbonate solution at 5-10° C.

This solution of methyl 1-aminopyrrolidine-2-carboxylate was slowly poured into a stirred solution of 3,3-dimethyl-butyraldehyde (1.10 g, 11 mmol) in methanol (100 mL) at 45° C. The mixture was allowed to cool to 25° C. and stirred for 16 h. The product was extracted with chloroform (6×100 mL), the combined organic layers were washed with brine (50 mL), dried over sodium sulfate, filtered and the solvent was removed in vacuo. The crude product was purified by flash column chromatography (Merck silica gel 60, 40-63 μM; 50% ethyl acetate in hexanes) to afford 1-(3,3-dimethyl-butylideneamino)-pyrrolidine-2-carboxylic acid methyl ester (1.5 g, 6.63 mmol, 60.2% over two steps) as a light red oil. LC-MS (ESI) calcd for C₁₂H₂₂N₂O₂ 226.17, found 227.3 [M+H⁺].

### i) 1-(3,3-Dimethyl-butylamino)-pyrrolidine-2-carboxylic acid methyl ester

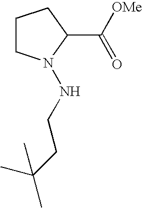

To a solution of 1-(3,3-dimethyl-butylideneamino)-pyrrolidine-2-carboxylic acid methyl ester (1.5 g, 6.6 mmol) in methanol (100 mL) was added acetic acid until a pH of 5-6 was reached, followed by sodium cyanoborohydride (1.04 g, 16.6 mmol). The resulting mixture was stirred at 25° C. for 2 h. The mixture was concentrated in vacuo to a volume of 20 mL. The reaction was quenched by addition of saturated aqueous sodium bicarbonate solution and extracted with ethyl acetate. The organic layer was washed with brine, dried over anhydrous magnesium sulfate, filtered and concentrated in vacuo to afford 1-(3,3-dimethyl-butylamino)-pyrrolidine-2-carboxylic acid methyl ester (1.3 g, 5.7 mmol, 86.4%) as a yellow oil, which was used directly in the next step.

### j) 1-{(3,3-Dimethyl-butyl)-[2-(7-methanesulfonylamino-1,1-dioxo-1,4-dihydro-1λ⁶-benzo[1,2,4]thiadiazin-3-yl)-acetyl]-amino}-pyrrolidine-2-carboxylic acid methyl ester

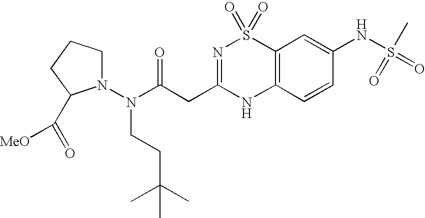

To a solution of (7-methanesulfonylamino-1,1-dioxo-1,4-dihydro-1λ⁶-benzo[1,2,4]thiadiazin-3-yl)-acetic acid (prepared as described in Example 1f, 0.146 g, 0.436 mmol) in N,N-dimethylformamide was added 1-(3,3-dimethyl-butylamino)-pyrrolidine-2-carboxylic acid methyl ester (0.30 g, 0.436 mmol) and a 1.0 M solution of N,N′-dicyclohexylcarbodiimide in dichloromethane (0.458 mL, 0.458 mmol). The reaction mixture was stirred for 2 h. Additional dichloromethane (2 mL) was added. The solid was filtered off and the filtrate was extracted with ethyl acetate and water. The organic layers were dried over sodium sulfate, filtered and the solvent was removed in vacuo. The crude product was purified by flash column chromatography (Merck silica gel 60, 40-63 μM; 80% ethyl acetate in hexanes) to afford 1-{(3,3-dimethyl-butyl)-[2-(7-methanesulfonylamino-1,1-dioxo-1,4-dihydro-1λ⁶-benzo[1,2,4]thiadiazin-3-yl)-acetyl]-amino}-pyrrolidine-2-carboxylic acid methyl ester (0.080 g, 0.147 mmol, 33.8%) as an off-white solid. LC-MS (ESI) calcd for C₂₂H₃₃N₅O₇S₂ 543.18, found 544.4 [M+H⁺].

### k) N-{3-[1-(3,3-Dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide

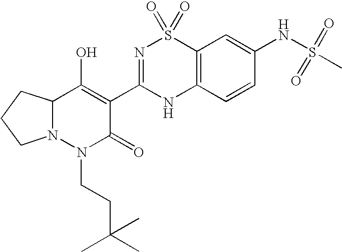

To a solution of 1-{(3,3-dimethyl-butyl)-[2-(7-methanesulfonylamino-1,1-dioxo-1,4-dihydro-1λ⁶-benzo[1,2,4]thiadiazin-3-yl)-acetyl]-amino}-pyrrolidine-2-carboxylic acid methyl ester (0.080 g, 0.147 mmol) in ethanol (2 mL) was added a 21% w/w solution of sodium ethoxide in ethanol (0.121 mL, 0.323 mmol) dropwise via syringe at 25° C. The reaction mixture was stirred for 1 h at 25° C., and then heated at 60° C. for 4 h. The reaction was quenched by addition of a 1.0 M aqueous hydrochloric acid solution and extracted with ethyl acetate. The organic layer was washed with brine, dried over sodium sulfate, filtered and concentrated in vacuo. The crude product was purified by flash column chromatography (Merck silica gel 60, 40-63 μM; 5% methanol in dichloromethane) to afford the desired product, N-{3-[1-(3,3-dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶ benzo[1,2,4]thiadiazin-7-yl}-methanesulfonamide (0.0229 g, 0.045 mmol, 30.5%), as a white solid. ¹H NMR (400 MHz, CDCl₃) δ: 1.00 (9H, s), 1.25-1.29 (1H, m), 1.50-1.71 (2H, m), 1.83-1.95 (2H, m), 2.15-2.25 (1H, m), 2.51-2.63 (1H, m), 2.75-2.92 (1H, m), 3.07 (3H, d, J=5.5 Hz), 3.16-3.22 (1H, m), 3.32-3.50 (2H, m), 3.84-4.23 (2H, m), 6.72 (1H, d, J=18.1 Hz), 7.18-7.25 (1H, m), 7.61-7.68 (2H, m). LC-MS (ESI) calcd for C₂₁H₂₉N₅O₆S₂ 511.16, found 512.5 [M+H⁺].

### EXAMPLE 2

### N-{3-[1-(3,3-Dimethyl-butyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methanesulfonamide

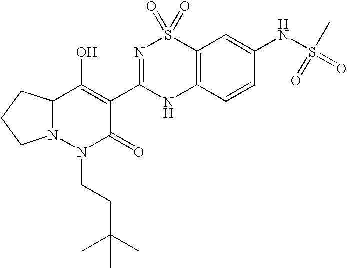

### a) 1-(3,3-Dimethyl-butylideneamino)-piperidine-2-carboxylic acid ethyl ester

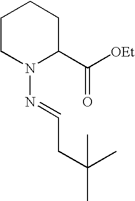

A 0.28 M solution of 1-oxa-2-aza-spiro[2.5]octane in toluene (prepared as described in Example 1g, 68 mL, 19.1 mmol) was added to piperidine-2-carboxylic acid ethyl ester (2.0 g, 12.7 mmol) and stirred under an inert atmosphere at 80° C. for 16 h. The mixture was cooled to 0° C. and a 1.0 M aqueous hydrochloric acid solution (30 mL) was added. The toluene phase was separated and extracted with an ice-cold 1.0 M aqueous hydrochloric acid solution (2×20 mL). The combined cold aqueous layers were washed with diethyl ether (2×30 mL), and carefully concentrated in vacuo at 25° C. to remove the remaining cyclohexanone. Crushed ice was added and the stirred acidic solution was neutralized by dropwise addition of saturated aqueous sodium bicarbonate solution (90 mL) at 5-10° C.

Half of this solution was slowly poured into a stirred solution of 3,3-dimethyl-butyraldehyde (0.636 g, 6.35 mmol) in methanol (63.5 mL) at 45° C. The mixture was allowed to cool to 25° C. and stirred for 16 h. The product was extracted with chloroform and the combined organic layers were washed with brine (30 mL), dried over sodium sulfate, filtered and concentrated in vacuo. The crude product was purified by flash column chromatography (Merck silica gel 60, 40-63 PM; 50% ethyl acetate/hexane) to afford the desired product, 1-(3,3-dimethyl-butylideneamino)-piperidine-2-carboxylic acid ethyl ester (1.58 g, 6.22 mmol, 97.5% over two steps), as a light yellow liquid. LC-MS (ESI) calcd for C₁₄H₂₆N₂O₂ 254.2, found 255.5 [M+H⁺].

### b) 1-(3,3-Dimethyl-butylamino)-piperidine-2-carboxylic acid ethyl ester

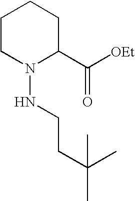

To a solution of 1-(3,3-dimethyl-butylideneamino)-piperidine-2-carboxylic acid ethyl ester (1.58 g, 6.35 mmol) in methanol (100 mL) was added acetic acid until a pH of 5-6 was reached, followed by sodium cyanoborohydride (0.993 g, 15.8 mmol). The reaction mixture was stirred at 25° C. for 2 h. The mixture was concentrated in vacuo to a volume of 20 mL. The reaction was quenched by addition of saturated aqueous sodium bicarbonate solution and extracted with ethyl acetate. The organic layer was washed with brine, dried over anhydrous magnesium sulfate, filtered and concentrated in vacuo to afford the desired product, 1-(3,3-dimethyl-butylamino)-piperidine-2-carboxylic acid ethyl ester (1.1 g, 4.29 mmol, 67.6%), as a colorless oil. LC-MS (ESI) calcd for C₁₄H₂₈N₂O₂ 256.22, found 257.2 [M+H⁺].

### c) 1-{(3,3-Dimethyl-butyl)-[2-(7-methanesulfonylamino-1,1-dioxo-1,4-dihydro-1λ⁶-benzo[1,2,4]thiadiazin-3-yl)-acetyl]-amino}-piperidine-2-carboxylic acid ethyl ester

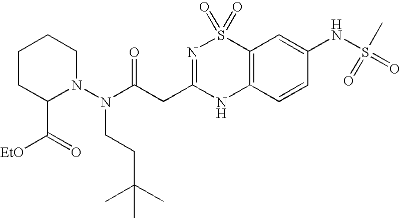

To a solution of (7-methanesulfonylamino-1,1-dioxo-1,4-dihydro-1λ⁶-benzo[1,2,4]thiadiazin-3-yl)-acetic acid (prepared as described in Example 1f, 0.070 g, 0.24 mmol) in N,N-dimethylformamide was added 1-(3,3-dimethyl-butylamino)-piperidine-2-carboxylic acid ethyl ester (0.062 g, 0.24 mmol) and a 1.0 M solution of N,N′-dicyclohexylcarbodiimide in dichloromethane (0.252 mL, 0.252 mmol). The reaction mixture was stirred for 2 h. Additional dichloromethane (2 mL) was added. The solid was filtered off and the filtrate was extracted with ethyl acetate and water. The organic layers were dried over sodium sulfate, filtered and the solvent was removed in vacuo. The crude product was purified by flash column chromatography (Merck silica gel 60, 40-63 μM; 80% ethyl acetate in hexanes) to afford the crude 1-{(3,3-dimethyl-butyl)-[2-(7-methanesulfonylamino-1,1-dioxo-1,4-dihydro-1λ⁶-benzo[1,2,4]thiadiazin-3-yl)-acetyl]-amino}-piperidine-2-carboxylic acid ethyl ester as an off-white solid, which was used directly in the next step. LC-MS (ESI) calcd for C₂₃H₃₅N₅O₇S₂ 557.2, found 558.2 [M+H⁺].

### d) N-{3-[1-(3,3-Dimethyl-butyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methane sulfonamide

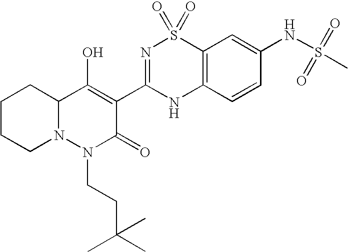

To a solution of 1-{(3,3-dimethyl-butyl)-[2-(7-methanesulfonylamino-1,1-dioxo-1,4-dihydro-1λ⁶-benzo[1,2,4]thiadiazin-3-yl)-acetyl]-amino}-piperidine-2-carboxylic acid ethyl ester (0.24 mmol) in ethanol (2 mL) was added a 21% w/w solution of sodium ethoxide in ethanol (0.197 mL, 0.528 mmol) dropwise via syringe at 25° C. The reaction mixture was stirred for 1 h at 25° C., and then heated at 60° C. for 4 h. The reaction was quenched by addition of a 1.0 M aqueous hydrochloric acid solution and extracted with ethyl acetate. The organic layer was washed with brine, dried over sodium sulfate and concentrated in vacuo. The crude product was purified by reverse phase HPLC to afford N-{3-[1-(3,3-dimethyl-butyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methanesulfonamide (10.6 mg, 0.020 mmol, 8.4% over two steps) as an white solid. ¹H NMR (400 MHz, CDCl₃) δ: 0.99 (9H, s), 1.27-1.33 (1H, m), 1.53-1.86 (6H, m), 2.41-2.51 (1H, m), 2.80-2.85 (1H, m), 2.89-3.01 (2H, m), 3.07 (3H, d, J=4.8 Hz), 3.83-4.15 (2H, m), 6.89 (1H, d, J=13.1 Hz), 7.18-7.24 (1H, m), 7.63-7.70 (2H, m). LC-MS (ESI) calcd for C₂₂H₃₁N₅O₆S₂ 525.17, found 526.4 [M+H⁺].

### EXAMPLE 3

### N-{3-[1-(4-Fluoro-benzyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methanesulfonamide

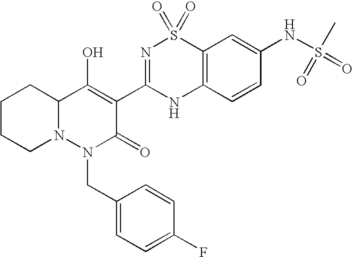

### a) 1-[(4-Fluoro-benzylidene)-amino]-piperidine-2-carboxylic acid ethyl ester

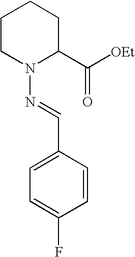

A 0.28 M solution of 1-oxa-2-aza-spiro[2.5]octane in toluene (prepared as described in Example 1g, 68 mL, 19.1 mmol) was added to piperidine-2-carboxylic acid ethyl ester (2.0 g, 12.7 mmol) and stirred under an inert atmosphere at 80° C. for 16 h. The mixture was cooled to 0° C. and a 1.0 M aqueous hydrochloric acid solution (30 mL) was added. The toluene phase was separated and extracted with an ice-cold 1.0 M aqueous hydrochloric acid solution (2×20 mL). The combined cold aqueous layers were washed with diethyl ether (2×30 mL), and carefully concentrated in vacuo at 25° C. to remove the remaining cyclohexanone. Crushed ice was added and the stirred acidic solution was neutralized by dropwise addition of saturated aqueous sodium bicarbonate solution (90 mL) at 5-10° C.

Half of this solution was slowly poured into a stirred solution of 4-fluoro-benzaldehyde (0.788 g, 6.35 mmol) in methanol (63.5 mL) at 45° C. The mixture was stirred at 25° C. for 16 h. The product was extracted with chloroform, the combined organic layers were washed with brine (30 mL), dried over sodium sulfate, filtered and concentrated in vacuo. The crude product was purified by flash column chromatography (Merck silica gel 60, 40-63 μM; 50% ethyl acetate in hexanes) to afford the desired product, 1-[(4-fluoro-benzylidene)-amino]-piperidine-2-carboxylic acid ethyl ester (1.3 g, 4.67 mmol, 73.6% over two steps), as a light yellow liquid. LC-MS (ESI) calcd for C₁₅H₁₉FN₂O₂ 278.14, found 279.3 [M+H⁺].

### b) 1-(4-Fluoro-benzylamino)-piperidine-2-carboxylic acid ethyl ester

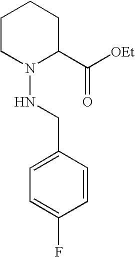

To a solution of 1-[(4-fluoro-benzylidene)-amino]-piperidine-2-carboxylic acid ethyl ester (1.3 g, 4.7 mmol) in methanol (100 mL) was added acetic acid until a pH of 5-6 was reached, followed by sodium cyanoborohydride (0.738 g, 11.75 mmol). The reaction mixture was stirred at 25° C. for 2 h. The mixture was concentrated in vacuo to a volume of 20 mL. The reaction was quenched by addition of saturated aqueous sodium bicarbonate solution and extracted with ethyl acetate. The organic layer was washed with brine, dried over anhydrous magnesium sulfate, filtered and concentrated in vacuo to afford the desired product, 1-(4-fluoro-benzylamino)-piperidine-2-carboxylic acid ethyl ester (1.05 g, 3.75 mmol, 79.8%), as a light yellow oil, which was used directly in the next step.

### c) 1-{(4-Fluoro-benzyl)-[2-(7-methanesulfonylamino-1,1-dioxo-1,4-dihydro-1λ⁶-benzo[1,2,4]thiadiazin-3-yl)-acetyl]-amino}-piperidine-2-carboxylic acid ethyl ester

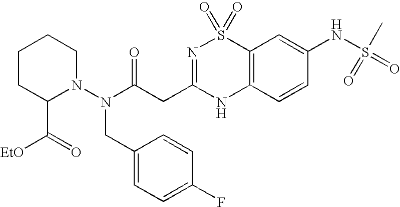

To a solution of (7-methanesulfonylamino-1,1-dioxo-1,4-dihydro-1λ⁶-benzo[1,2,4]thiadiazin-3-yl)-acetic acid (prepared as described in Example 1f, 0.080 g, 0.24 mmol) in N,N-dimethylformamide was added 1-(4-fluoro-benzylamino)-piperidine-2-carboxylic acid ethyl ester (0.067 g, 0.24 mmol) and a 1.0 M solution of N,N′-dicyclohexylcarbodiimide in dichloromethane (0.252 mL, 0.252 mmol). The reaction mixture was stirred for 2 h. Additional dichloromethane (2 mL) was added. The solid was filtered off and the filtrate was extracted with ethyl acetate and water. The organic layers were dried over sodium sulfate, filtered and the solvent was removed in vacuo. The crude product was purified by flash column chromatography (Merck silica gel 60, 40-63 μM; 80% ethyl acetate in hexanes) to afford the crude 1-{(4-fluoro-benzyl)-[2-(7-methanesulfonylamino-1,1-dioxo-1,4-dihydro-1λ⁶-benzo[1,2,4]thiadiazin-3-yl)-acetyl]-amino}-piperidine-2-carboxylic acid ethyl ester as an off-white solid, which was used directly in the next step. LC-MS (ESI) calcd for C₂₅H₃₀FN₅O₇S₂ 595.16, found 596.4 [M+H⁺].

### d) N-{3-[1-(4-Fluoro-benzyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methane sulfonamide

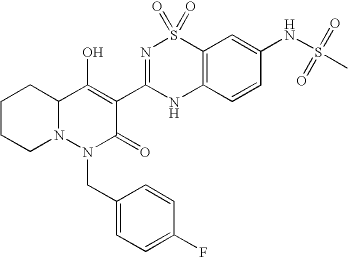

To a solution of crude 1-{(4-fluoro-benzyl)-[2-(7-methanesulfonylamino-1,1-dioxo-1,4-dihydro-1λ⁶-benzo[1,2,4]thiadiazin-3-yl)-acetyl]-amino}-piperidine-2-carboxylic acid ethyl ester (0.24 mmol) in ethanol (2 mL) was added a 21% w/w solution of sodium ethoxide in ethanol (0.197 mL, 0.528 mmol) dropwise via syringe at 25° C. The reaction mixture was stirred for 1 h at 25° C., and then heated at 60° C. for 4 h. The reaction was quenched by addition of a 1.0 M aqueous hydrochloric acid solution and extracted with ethyl acetate. The organic layer was washed with brine, dried over sodium sulfate, filtered and concentrated in vacuo. The crude product was purified by reverse phase HPLC to afford the desired product, N-{3-[1-(4-fluoro-benzyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methanesulfonamide (0.0125 g, 0.0228 mmol, 9.5% over two steps), as a light yellow solid. ¹H NMR (400 MHz, CDCl₃) δ: 1.21-1.32 (1H, m), 1.52-1.77 (5H, m), 2.28-2.44 (1H, m), 2.83-2.93 (2H, m), 3.07 (3H, d, J=4.9 Hz), 4.03-4.05 (1H, m), 4.12-4.40 (1H, m), 4.93-5.07 (1H, m), 6.97-7.12 (3H, m), 7.19-7.25 (1H), 7.33-7.41 (2H, m), 7.62-7.71 (2H, m). LC-MS (ESI) calcd for C₂₃H₂₄FN₅O₆S₂ 549.12, found 550.5 [M+H⁺].

### EXAMPLE 4

### N-{3-[1-(3,3-Dimethyl-butyl)-4-hydroxy-4-a-methyl-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide

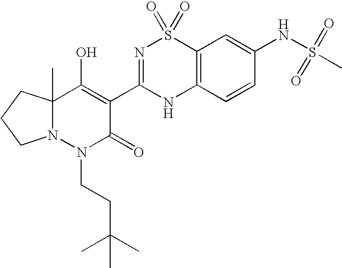

### a) 2-Methyl-pyrrolidine-1,2-dicarboxylic acid 1-benzyl ester 2-methyl ester

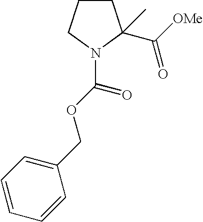

To a solution of N-benzyloxycarbonylproline methyl ester (2.28 g, 8.67 mmol) in tetrahydrofuran (12 mL) at −78° C. was added a 2.0 M solution of lithium diisopropylamide in tetrahydrofuran (4.8 mL, 9.6 mmol) under a nitrogen atmosphere. After stirring for 15 min, methyl iodide (4.95 g, 34.8 mmol) was added and the reaction mixture was stirred for 4 h at −78° C. The solution was allowed to warm to 25° C. before it was poured into a 1.0 M aqueous hydrochloric acid solution and extracted with ethyl acetate. The organic layer was dried over sodium sulfate, filtered, and concentrated in vacuo to afford the crude product. Further purification by flash column chromatography (Teldyne Isco RediSep Column; 0-50% ethyl acetate in hexanes) afforded the desired product, 2-methyl-pyrrolidine-1,2-dicarboxylic acid 1-benzyl ester 2-methyl ester (1.225 g, 4.42 mmol, 51%), as a yellow oil. LC-MS (ESI) calcd for C₁₅H₁₉NO₄ 277.13, found 278.2 [M+H⁺].

### b) 2-Methyl-pyrrolidine-2-carboxylic acid methyl ester

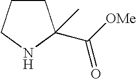

To a solution of 2-methyl-pyrrolidine-1,2-dicarboxylic acid 1-benzyl ester 2-methyl ester (0.277 g, 1 mmol) in methanol (10 mL) under a nitrogen atmosphere was added 10% palladium on carbon (0.106 g). The mixture was degassed while stirring and the flask was charged with hydrogen gas via balloon. The mixture was stirred at 25° C. for 5 h. The mixture was filtered through Celite (rinsed with methanol) and the filtrate was concentrated in vacuo to afford the desired product, 2-methyl-pyrrolidine-2-carboxylic acid methyl ester (139 mg, 0.97 mmol, 97%) as a colorless oil. LC-MS (ESI) calcd for C₇H₁₃NO₂ 143.09, found 144.2 [M+H⁺].

### c) 1-(3,3-Dimethyl-butylideneamino)-2-methyl-pyrrolidine-2-carboxylic acid methyl ester

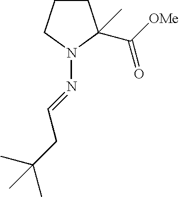

A 0.28 M solution of 1-oxa-2-aza-spiro[2.5]octane in toluene (prepared as described in Example 1g, 55 mL, 15.5 mmol) was added to 2-methyl-pyrrolidine-2-carboxylic acid methyl ester (0.738 g, 5.15 mmol) and stirred under an inert atmosphere at 80° C. for 16 h. The mixture was cooled to 0° C. and a 1.0 M aqueous hydrochloric acid solution (15 mL) was added. The toluene phase was separated and extracted with an ice-cold 1.0 M aqueous hydrochloric acid solution (2×10 mL). The combined cold aqueous layers were washed with diethyl ether (2×10 mL), and carefully concentrated in vacuo at 25° C. to remove the remaining cyclohexanone. Crushed ice (30 g) was added and the stirred acidic solution was neutralized by dropwise addition of saturated aqueous sodium bicarbonate solution at 5-10° C.

This solution was slowly poured into a stirred solution of 3,3-dimethyl-butyraldehyde (0.516 g, 5.15 mmol) in methanol (50 mL) at 45° C. The mixture was allowed to cool to 25° C. and stirred for 16 h. Water was added and the product was extracted with chloroform (6×50 mL), the combined organic layers were washed with brine (50 mL), dried over sodium sulfate, filtered and the solvent was removed in vacuo. Further purification by flash column chromatography (Teledyne Isco RediSep Column; 0-50% ethyl acetate in hexanes) afforded the desired product, 1-(3,3-dimethyl-butylideneamino)-2-methyl-pyrrolidine-2-carboxylic acid methyl ester (485 mg, 2.019 mmol, 39.1% over two steps), as a light yellow oil. LC-MS (ESI) calcd for C₁₃H₂₄N₂O₂ 240.18, found 241.4 [M+H⁺].

### d) 1-(3,3-Dimethyl-butylamino)-2-methyl-pyrrolidine-2-carboxylic acid methyl ester

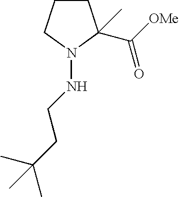

To a solution of 1-(3,3-dimethyl-butylideneamino)-2-methyl-pyrrolidine-2-carboxylic acid methyl ester (0.485 g, 2.01 mmol) in methanol (100 mL) was added acetic acid until a pH of 5-6 was reached, followed by sodium cyanoborohydride (0.302 g, 4.81 mmol). The reaction mixture was stirred at 25° C. for 4 h. The mixture was poured into a saturated aqueous sodium bicarbonate solution and was extracted with ethyl acetate. The organic layer was dried over anhydrous magnesium sulfate, filtered and concentrated in vacuo to afford the desired product, 1-(3,3-dimethyl-butylamino)-2-methyl-pyrrolidine-2-carboxylic acid methyl ester (0.356 g, 1.47 mmol, 73.4%) as a colorless oil, which was used directly in the next step. LC-MS (ESI) calcd for C₁₃H₂₆N₂O₂ 242.20, found 243.4 [M+H⁺].

### e) 1-{(3,3-Dimethyl-butyl)-[2-(7-methanesulfonylamino-1,1-dioxo-1,4-dihydro-1λ⁶-benzo[1,2,4]thiadiazin-3-yl)-acetyl]-amino}-2-methyl-pyrrolidine-2-carboxylic acid methyl ester

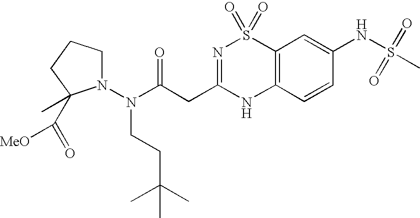

To a solution of (7-methanesulfonylamino-1,1-dioxo-1,4-dihydro-1λ⁶-benzo[1,2,4]thiadiazin-3-yl)-acetic acid (prepared as described in Example 1f, 0.166 g, 0.500 mmol) in N,N-dimethylformamide (25 mL) was added 1-(3,3-dimethyl-butylamino)-2-methyl-pyrrolidine-2-carboxylic acid methyl ester (0.122 g, 0.500 mmol) and a 1.0 M solution of N,N′-dicyclohexylcarbodiimide in dichloromethane (0.525 mL, 0.525 mmol). The reaction mixture was stirred for 3 h. The solid was filtered off and the filtrate was concentrated in vacuo. The crude product was purified by flash column chromatography (Teledyne Isco RediSep Column; 5% methanol in dichloromethane) to afford the desired product, 1-{(3,3-dimethyl-butyl)-[2-(7-methanesulfonylamino-1,1-dioxo-1,4-dihydro-1λ⁶-benzo[1,2,4]thiadiazin-3-yl)-acetyl]-amino}-2-methyl-pyrrolidine-2-carboxylic acid methyl ester (0.249 g, 0.447 mmol, 89.3%), as a yellow oil. LC-MS (ESI) calcd for C₂₃H₃₅N₅O₇S₂ 557.20, found 558.3 [M+H⁺].

### f) N-{3-[1-(3,3-Dimethyl-butyl)-4-hydroxy-4a-methyl-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide

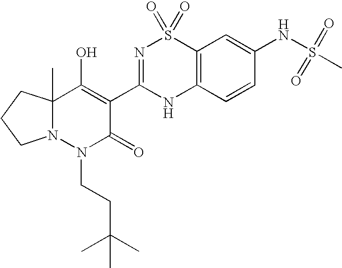

To a solution of 1-{(3,3-dimethyl-butyl)-[2-(7-methanesulfonylamino-1,1-dioxo-1,4-dihydro-1λ⁶-benzo[1,2,4]thiadiazin-3-yl)-acetyl]-amino}-2-methyl-pyrrolidine-2-carboxylic acid methyl ester (0.249 g, 0.447 mmol) in ethanol (4 mL) was added a 21% w/w solution of sodium ethoxide in ethanol (0.368 mL, 0.982 mmol) dropwise via syringe at 25° C. The reaction mixture was stirred for 1 h at 25° C., and then heated at 60° C. for 4 h. The reaction was quenched by addition of a 1.0 M aqueous hydrochloric acid solution upon which a solid precipitated. The solid was collected by vacuum filtration, washed with water and extracted with ethyl acetate. The organic layer was washed with brine, dried over sodium sulfate and concentrated in vacuo to afford the desired product, N-{3-[1-(3,3-dimethyl-butyl)-4-hydroxy-4a-methyl-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide (0.1129 g, 0.215 mmol, 48.3%) as an off-white solid. ¹H NMR (400 MHz, DMSO-d₆) δ: 0.94 (9H, s), 1.18-1.24 (3H, m), 1.41-1.49 (3H, m), 1.55-1.76 (3H, m), 2.53-2.59 (1H, m), 2.78-2.84 (1H, m), 3.04 (3H, s), 3.19-3.43 (2H, m), 3.76-3.86 (1H, m), 7.46-7.53 (3H, m), 10.08 (1H, bs). LC-MS (ESI) calcd for C₂₂H₃₁N₅O₆S₂ 525.17, found 526.3.

### EXAMPLE 5

### N-{3-[7-(3,3-Dimethyl-butyl)-10-hydroxy-6-methyl-8-oxo-6,7-diaza-spiro[4,5]dec-9-en-9-yl]-1,1-dioxo-1,4-dihydro-1λ⁶-benzo[1,2,4]thiadiazin-7-yl}-methanesulfonamide trifluoroacetic acid salt

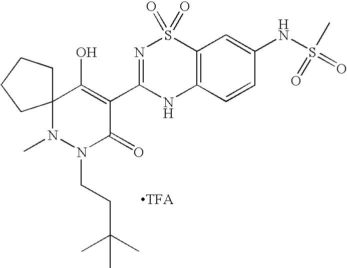

### a) 1-Hydrazino-cyclopentanecarboxylic acid methyl ester

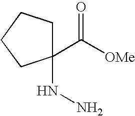

1-Amino-cyclopentanecarboxylic acid methyl ester hydrochloride (2.00 g, 11.13 mmol) was dissolved in aqueous saturated sodium bicarbonate solution (40 mL). The solution was extracted with ethyl acetate (3×40 mL). The organic layer was washed with aqueous saturated brine solution (20 mL), dried over magnesium sulfate, filtered, and concentrated in vacuo to afford a clear oil. The oil was mixed with a 0.28 M solution of 1-oxa-2-aza-spiro[2.5]octane in toluene (prepared as described in Example 1g, 100 mL, 28 mmol). The reaction was stirred at 80° C. for 18 h. The mixture was then cooled to 0° C. and acidified via dropwise addition of 1.0 M aqueous hydrochloric acid solution to pH 2-3. The toluene phase was separated and extracted with 1.0 M aqueous hydrochloric acid solution (2×20 mL). The aqueous phases were combined and washed with ethyl ether (20 mL). The aqueous solution was cooled to 0° C. and crushed ice was added into the solution. It was then neutralized with aqueous saturated sodium bicarbonate solution to pH 8. The resulting solution containing the crude product, 1-hydrazino-cyclopentanecarboxylic acid methyl ester, was used without any further purification in the next step.

### b) 1-[N′-(3,3-Dimethyl-butylidene)-hydrazino]-cyclopentanecarboxylic acid methyl ester

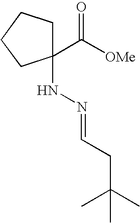

3,3-Dimethyl-butyraldehyde (0.98 mL, 7.79 mmol) was dissolved in methanol (70 mL). The aqueous solution of 1-hydrazino-cyclopentanecarboxylic acid methyl ester (crude from Example 5a, ˜7.79 mmol, based on 70% yield for the previous step) was added. The reaction was stirred at 25° C. for 18 h. The solution was concentrated in vacuo to remove the methanol. The remaining aqueous solution was extracted with ethyl acetate (3×60 mL). The organic layers were combined and washed with aqueous saturated brine solution (20 mL), dried over magnesium sulfate, filtered, and concentrated in vacuo to afford a clear oil. Purification by flash column chromatography (Teledyne Isco RediSep Column; 0-20% ethyl acetate in hexanes) afforded the desired product, 1-[N′-(3,3-dimethyl-butylidene)-hydrazino]-cyclopentanecarboxylic acid methyl ester (1.70 g, 7.09 mmol, 91%) as a clear oil. ¹H NMR (400 MHz, CDCl₃) δ: 0.94 (9H, s), 1.74-1.86 (4H, m), 1.95-2.14 (6H, m), 3.68 (3H, s), 7.18 (1H, t, J=6.4 Hz). LC-MS (ESI) calcd for C₁₃H₂₄N₂O₂ 240.34, found 241.3 [M+H⁺].

### c) (1-[N′-(3,3-Dimethyl-butylidene)-N-methyl-hydrazino]-cyclopentanecarboxylic acid methyl ester

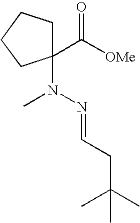

1-[N′-(3,3-Dimethyl-butylidene)-hydrazino]-cyclopentanecarboxylic acid methyl ester (0.67 g, 2.80 mmol) was dissolved in anhydrous methanol (5 mL). A 0.5 M solution of sodium methoxide in methanol (5.6 mL, 2.80 mmol) was added dropwise to the above solution. The mixture was stirred at 25° C. for 10 min. Iodomethane (0.77 mL, 12.32 mmol) was added to the mixture. The reaction was stirred at 40° C. for 96 h. The solution was then concentrated in vacuo and the residue was dissolved in ethyl acetate (30 mL). The solution was further washed with 1.0 M aqueous hydrochloric acid solution (2×20 mL), aqueous saturated brine solution (20 mL), dried over magnesium sulfate, filtered, and concentrated in vacuo to afford a clear oil. Purification by flash column chromatography (Teledyne Isco RediSep Column; 0-15% ethyl acetate in hexanes) afforded the desired product, (1-[N′-(3,3-dimethyl-butylidene)-N-methyl-hydrazino]-cyclopentanecarboxylic acid methyl ester (0.60 g, 2.35 mmol, 84%) as a clear oil. ¹H NMR (400 MHz, CDCl₃) δ: 0.94 (9H, s), 1.71-1.77 (4H, m), 2.10-2.23 (6H, m), 2.70 (3H, s), 3.67 (3H, s), 6.68 (1H, bs). LC-MS (ESI) calcd for C₁₄H₂₆N₂O₂ 254.37, found 255.1 [M+H⁺].

### d) 1-[N′-(3,3-Dimethyl-butyl)-N-methyl-hydrazino]-cyclopentanecarboxylic acid methyl ester

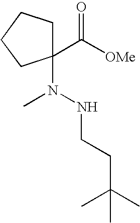

(1-[N′-(3,3-Dimethyl-butylidene)-N-methyl-hydrazino]-cyclopentanecarboxylic acid methyl ester (0.24 g, 0.92 mmol) was dissolved in methanol (10 mL) and acetic acid (2 mL). Sodium cyanoborohydride (0.15 g, 2.30 mmol) was added to the above solution. The reaction was stirred at 25° C. for 2 h. The reaction was quenched via the addition of saturated aqueous sodium bicarbonate solution (20 mL). The mixture was extracted with ethyl acetate (3×30 mL). The organic layers were combined, further washed with saturated aqueous brine solution (20 mL), dried over magnesium sulfate, filtered, and concentrated in vacuo to afford a golden oil. Purification by flash column chromatography (Teledyne Isco RediSep Column; 0-30% ethyl acetate in hexanes) afforded the desired product, 1-[N′-(3,3-dimethyl-butyl)-N-methyl-hydrazino]-cyclopentanecarboxylic acid methyl ester (0.17 g, 0.66 mmol, 72%) as a clear oil. ¹H NMR (400 MHz, CDCl₃) δ: 0.92 (9H, s), 1.31-1.35 (2H, m), 1.71-1.72 (4H, m), 1.86-2.11 (4H, m), 2.57 (3H, s), 2.74-2.79 (2H, m), 3.71 (3H, s). LC-MS (ESI) calcd for C₁₄H₂₈N₂O₂ 256.38, found 257.1 [M+H⁺].

### e) N-{3-[7-(3,3-Dimethyl-butyl)-10-hydroxy-6-methyl-8-oxo-6,7-diaza-spiro[4.5]dec-9-en-9-yl]-1,1-dioxo-1,4-dihydro-1%-benzo[1,2,4]thiadiazin-7-yl}-methanesulfonamide trifluoroacetic acid salt

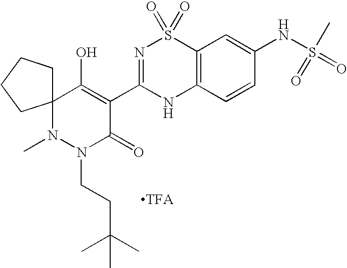

1-[N′-(3,3-Dimethyl-butyl)-N-methyl-hydrazino]-cyclopentanecarboxylic acid methyl ester (0.156 g, 0.61 mmol) was dissolved in anhydrous N,N-dimethylformamide (4 mL). (7-Methanesulfonylamino-1,1-dioxo-1,4-dihydro-1λ⁶-benzo[1,2,4]thiadiazin-3-yl)-acetic acid (prepared as described in Example 1f, 203 mg, 0.61 mmol) was added followed by a 1.0 M solution of N,N-dicyclohexylcarbodiimide in dichloromethane (0.67 mL, 0.67 mmol). The mixture was stirred at 25° C. for 16 h. The reaction was concentrated in vacuo to dryness. The residue was dissolved in ethanol (10 mL). A 21% w/w solution of sodium ethoxide in ethanol (0.45 mL, 1.21 mmol) was added to the above solution. The reaction was stirred at 60° C. for 4 h before it was quenched with 1.0 M aqueous hydrochloric acid solution (20 mL). The mixture was extracted with ethyl acetate (3×30 mL). The organic layers were combined and washed with brine (20 mL), dried over magnesium sulfate, filtered, and concentrated in vacuo to afford a golden oil. Purification by HPLC (Column Luna 5μ C18 (2) Å AXIA 150×21.2 mm, 5 micron, 30%-100% in 12 min @30 mL/min flow rate, 0.05% trifluoroacetic acid in acetonitrile/0.05% trifluoroacetic acid in water) afforded the desired product, N-{3-[7-(3,3-dimethyl-butyl)-10-hydroxy-6-methyl-8-oxo-6,7-diaza-spiro[4.5]dec-9-en-9-yl]-1,1-dioxo-1,4-dihydro-1λ⁶-benzo[1,2,4]thiadiazin-7-yl}-methane sulfonamide (0.26 g, 0.49 mmol, 81%) as a white powder. M.p. 203.8-205.7° C. ¹H NMR (400 MHz, DMSO-d₆) δ: 0.95 (9H, s), 1.38-1.84 (6H, m), 1.91-2.08 (2H, m), 2.10-2.28 (2H, m), 2.53 (3H, s), 3.06 (3H, s), 3.75-3.96 (2H, m), 7.49-7.60 (3H, m), 10.14 (1H, s), 13.31 (1H, s). LC-MS (ESI) calcd for C₂₃H₃₃N₅O₆S₂ 539.67, found 540.3 [M+H⁺].

### EXAMPLE 6

### N-{3-[4-Hydroxy-1-(3-methyl-butyl)-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide

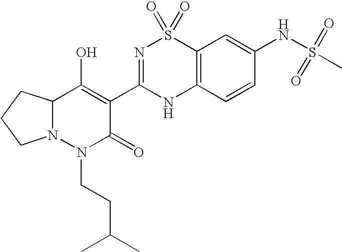

### a) 1-(3-Methyl-butylideneamino)-pyrrolidine-2-carboxylic acid methyl ester

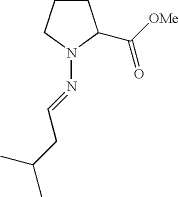

3-Methyl-butyraldehyde (935 μL, 8.75 mmol) was dissolved in methanol (87 mL) and heated at 45° C. for 20 min before adding a solution of methyl 1-aminopyrrolidine-2-carboxylate (prepared as described in Example 1h, 1.25 g, 8.65 mmol) in water (117 mL). The mixture was stirred for 10 min at 45° C. then was allowed to cool to 25° C. and was stirred for another 16 h. The mixture was diluted with water (100 mL) and extracted with chloroform (3×250 mL). The organic layers were dried over sodium sulfate, filtered and concentrated in vacuo to afford the crude product, 1-(3-methyl-butylideneamino)-pyrrolidine-2-carboxylic acid methyl ester (237 mg, 1.12 mmol, 12.7%) as a brown oil that solidified upon standing, which was used without any further purification in the next step. ¹H NMR (400 MHz, CDCl₃) δ: 1.00 (6H, d, J=6.1 Hz), 1.27 (2H, t, J=7.0 Hz), 1.71-1.76 (1H, m), 1.85-1.91 (2H, m), 2.06 (2H, s), 2.18-2.26 (1H, m), 2.30 (1H, d, J=2.4 Hz), 2.31-2.37 (2H, m), 3.50 (1H, s), 4.13 (1H, q, J=7.2 Hz), 9.74 (1H, s).

### b) 1-(3-Methyl-butylamino)-pyrrolidine-2-carboxylic acid methyl ester

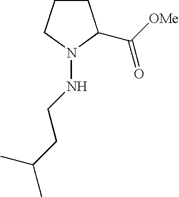

1-(3-Methyl-butylideneamino)-pyrrolidine-2-carboxylic acid methyl ester (200 mg, 0.942 mmol) was suspended in methanol (7.5 mL) and glacial acetic acid (20 drops) was added until pH 5-6 was reached. Sodium cyanoborohydride (0.147 g, 2.35 mmol) was added and the mixture was stirred at 25° C. for 16 h. The mixture was poured into a saturated aqueous sodium bicarbonate solution (10 mL) and was extracted with ethyl acetate (3×15 mL). The combined organic layers were dried over sodium sulfate, filtered, and concentrated in vacuo to afford the crude product, 1-(3-methyl-butylamino)-pyrrolidine-2-carboxylic acid methyl ester (179 mg, 0.836 mmol, 88.7%) as a brown oil, which was used without any further purification in the next step.

### c) 1-[[2-(7-Methanesulfonylamino-1,1-dioxo-1,4-dihydro-1λ⁶ benzo[1,2,4]thiadiazin-3-yl)-acetyl]-(3-methyl-butyl)-amino]-pyrrolidine-2-carboxylic acid methyl ester

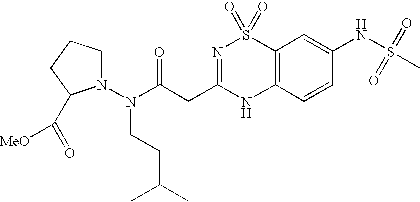

To a solution of 1-(3-methyl-butylamino)-pyrrolidine-2-carboxylic acid methyl ester (174 mg, 0.811 mmol) in N,N-dimethylformamide (4 mL) was added (7-methanesulfonylamino-1,1-dioxo-1,4-dihydro-1λ⁶-benzo[1,2,4]thiadiazin-3-yl)-acetic acid (prepared as described in Example 1f, 270.6 mg, 0.811 mmol). After sonication, once everything was dissolved, a 1.0 M solution of N,N-dicyclohexylcarbodiimide in dichloromethane was added and the mixture was stirred at 25° C. for 16 h. Dichloromethane (40 mL) was added and the precipitate was removed by vacuum filtration. The filtrate was diluted with dichloromethane (30 mL) and washed with water (100 mL). The aqueous layer was extracted with ethyl acetate and the combined organic layers were dried over magnesium sulfate, filtered and concentrated in vacuo. The residue was purified by flash column chromatography (Teledyne Isco RediSep Column; 0-10% methanol in dichloromethane) to afford the crude product, 1-[[2-(7-methanesulfonylamino-1,1-dioxo-1,4-dihydro-1λ⁶-benzo[1,2,4]thiadiazin-3-yl)-acetyl]-(3-methyl-butyl)-amino]-pyrrolidine-2-carboxylic acid methyl ester (174 mg, 0.32 mmol, 39.4%), which was used without any further purification in the next step.

### d) N-{3-[4-Hydroxy-1-(3-methyl-butyl)-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide

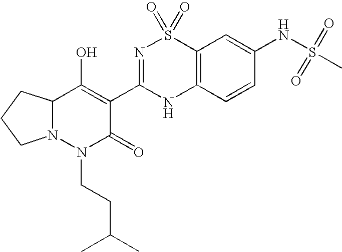

To a solution of the crude 1-[[2-(7-methanesulfonylamino-1,1-dioxo-1,4-dihydro-1λ⁶-benzo[1,2,4]thiadiazin-3-yl)-acetyl]-(3-methyl-butyl)-amino]-pyrrolidine-2-carboxylic acid methyl ester (174 mg, 0.32 mmol) in ethanol (3.2 mL) was added a 21% w/w solution of sodium ethoxide in ethanol (235 mg, 0.72 mmol). The mixture was heated at 60° C. for 3 h. Upon cooling, water (4 mL) was added and the precipitate was removed by vacuum filtration. The aqueous layer was extracted with ethyl acetate, dried over magnesium sulfate, filtered, and concentrated in vacuo. Further purification by prep-HPLC afforded the desired product, N-{3-[4-hydroxy-1-(3-methyl-butyl)-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide (16 mg, 0.032 mmol, 10%) as a solid. ¹H NMR (400 MHz, CDCl₃) δ: 0.98 (6H, d, J=3.9 Hz), 1.51-1.69 (4H, m), 1.77-1.92 (2H, m), 2.51-2.63 (1H, m), 2.76-2.87 (1H, m), 3.08 (3H, s), 3.15-3.22 (1H, m), 3.30-3.42 (1H, m), 3.83-4.02 (1H, m), 4.14-4.17 (1H, m), 6.50 (1H, d, J=15.8 Hz), 7.19-7.25 (1H, m), 7.60-7.66 (2H, m), 13.70 (1H, s), 13.88 (1H, s). LC-MS (ESI) calcd for C₂₀H₂₇N₅O₆S₂ 497.14, found 498.1 [M+H⁺].

### EXAMPLE 7

### N-{3-[1-(4-Fluoro-benzyl)-4-hydroxy-2-oxo-1,2,4a,5,6,7-hexahydro-pyrrolo[1,2-b]pyridazin-3-yl]-1,1-dioxo-1,4-dihydro-1λ⁶ benzo[1,2,4]thiadiazin-7-yl}-methanesulfonamide

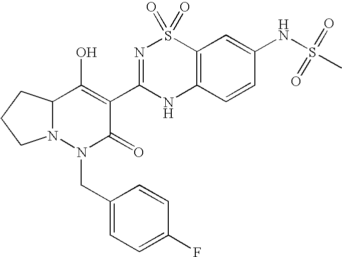

### a) 1-[(4-Fluoro-benzylidene)-amino]-pyrrolidine-2-carboxylic acid methyl ester

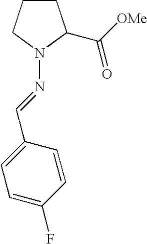

A 0.28 M solution of 1-oxa-2-aza-spiro[2.5]octane in toluene (prepared as described in Example 1g, 55 mL, 15.48 mmol) was added to DL-proline methyl ester (1 g, 7.74 mmol) and stirred under an inert atmosphere at 80° C. for 16 h. The mixture was cooled to 0° C. and a 1.0 M aqueous hydrochloric acid solution (10 mL) was added. The toluene phase was separated and extracted with an ice-cold 1.0 M aqueous hydrochloric acid solution (2×10 mL). The combined cold aqueous layers were washed with diethyl ether (2×10 mL), and carefully concentrated in vacuo at 25° C. to remove the remaining cyclohexanone. Crushed ice (70 g) was added and the stirred acidic solution was neutralized by dropwise addition of saturated aqueous sodium bicarbonate solution (25 mL) at 5-10° C.

This solution was slowly poured into a stirred solution of 4-fluoro-benzaldehyde (0.961 g, 7.74 mmol) in methanol (70 mL) at 45° C. The mixture was allowed to cool to 25° C. and stirred for 2 h. The product was extracted with chloroform (6×100 mL), the combined organic layers were washed with brine (50 mL), dried over sodium sulfate, filtered and concentrated in vacuo. The crude product was purified by flash column chromatography (Merck silica gel 60, 40-63 μM; 50% ethyl acetate in hexanes) to afford the desired product, 1-[(4-fluoro-benzylidene)-amino]-pyrrolidine-2-carboxylic acid methyl ester (1.217 g, 4.86 mmol, 62.7% over two steps) as a light red oil. ¹H NMR (400 MHz, CDCl₃) δ: 1.92-1.98 (1H, m), 2.09-2.36 (3H, m), 2.42 (1H, t, J=6.6 Hz), 3.31-3.37 (1H, m), 3.61-3.66 (1H, m), 3.83 (3H, s), 4.37 (1H, dd, J₁=8.7 Hz, J₂=3.2 Hz), 7.05 (2H, t, J=9.0 Hz), 7.56 (2H, q, J=4.6 Hz). LC-MS (ESI) calcd for C₁₃H₁₅FN₂O₂ 250.11, found 251.3 [M+H⁺].

### b) 1-(4-Fluoro-benzylamino)-pyrrolidine-2-carboxylic acid methyl ester

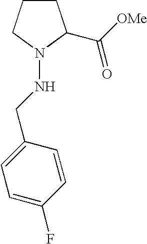

To a solution of 1-[(4-fluoro-benzylidene)-amino]-pyrrolidine-2-carboxylic acid methyl ester (1.17 g, 4.67 mmol) in methanol (20 mL) was added acetic acid until a pH of 5-6 was reached, followed by sodium cyanoborohydride (1.234 g, 19.64 mmol). The resulting mixture was stirred at 25° C. for 22 h. The reaction was quenched by addition of saturated aqueous sodium bicarbonate solution and extracted with ethyl acetate. The organic layer was washed with brine, dried over anhydrous magnesium sulfate, filtered and concentrated in vacuo to afford 1-(4-fluoro-benzylamino)-pyrrolidine-2-carboxylic acid methyl ester (0.863 g, 3.42 mmol, 73.2%) as a colorless oil, which was used directly in the next step. ¹H NMR (400 MHz, CDCl₃) δ: 1.89-2.02 (3H, m), 2.14-2.21 (2H, m), 2.75-2.81 (1H, m), 3.34-3.39 (1H, m), 3.59-3.63 (1H, m), 3.79 (3H, s), 3.83 (2H, s), 7.05 (2H, t, J=8.5 Hz), 7.34-7.38 (2H, m). LC-MS (ESI) calcd for C₁₃H₁₇FN₂O₂ 252.13, found 253.3 [M+H⁺].

### c) 1-{(4-Fluoro-benzyl)-[2-(7-methanesulfonylamino-1,1-dioxo-1,4-dihydro-1λ⁶-benzo[1,2,4]thiadiazin-3-yl)-acetyl]-amino}-pyrrolidine-2-carboxylic acid methyl ester

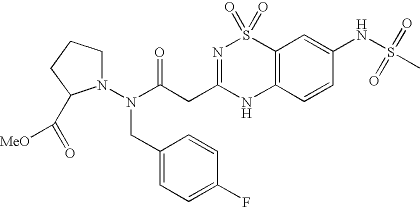

To a solution of (7-methanesulfonylamino-1,1-dioxo-1,4-dihydro-1λ⁶-benzo[1,2,4]thiadiazin-3-yl)-acetic acid (prepared as described in Example 1f, 0.0705 g, 0.212 mmol) in N,N-dimethylformamide was added 1-(4-fluoro-benzylamino)-pyrrolidine-2-carboxylic acid methyl ester (0.054 g, 0.212 mmol) and a 1.0 M solution of N,N′-dicyclohexylcarbodiimide in dichloromethane (0.223 mL, 0.223 mmol). The reaction mixture was stirred for 2 h. Additional dichloromethane (2 mL) was added. The solid was filtered off and the filtrate was extracted with ethyl acetate and water. The organic layers were dried over sodium sulfate, filtered and concentrated in vacuo. The crude product was purified by flash column chromatography (Merck silica gel 60, 40-63 μM; 5% methanol in dichlormethane) to afford the desired product, 1-{(4-fluoro-benzyl)-[2-(7-methanesulfonylamino-1,1-dioxo-1,4-dihydro-1λ⁶-benzo[1,2,4]thiadiazin-3-yl)-acetyl]-amino}-pyrrolidine-2-carboxylic acid methyl ester as a light yellow oil which was used directly in the next step. LC-MS (ESI) calcd for C₂₃H₂₆FN₅O₇S₂ 567.13, found 568.4 [M+H⁺].

### d) N-{3-[1-(4-Fluoro-benzyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methane sulfonamide

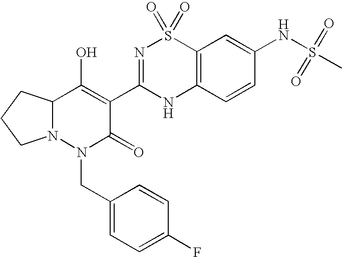

To a solution of 1-{(4-fluoro-benzyl)-[2-(7-methanesulfonylamino-1,1-dioxo-1,4-dihydro-1λ⁶-benzo[1,2,4]thiadiazin-3-yl)-acetyl]-amino}-pyrrolidine-2-carboxylic acid methyl ester (crude from Example 7c, 0.212 mmol) in ethanol (2 mL) was added a 21% w/w solution of sodium ethoxide in ethanol (0.174 mL, 0.466 mmol) dropwise via syringe at 25° C. The reaction mixture was stirred for 1 h at 25° C., and then heated at 60° C. for 3 h. The reaction was quenched by addition of a 1.0 M aqueous hydrochloric acid solution and extracted with ethyl acetate. The organic layer was washed with brine, dried over sodium sulfate, filtered and concentrated in vacuo. The crude product was purified by flash column chromatography (Merck silica gel 60, 40-63 μM; 5% methanol in dichloromethane) to afford the desired product, N-{3-[1-(4-fluoro-benzyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide (0.013 g, 0.024 mmol, 11.3% over 2 steps) as a light yellow solid. ¹H NMR (400 MHz, DMSO-d₆) δ: 1.65-1.74 (2H, m), 1.93-2.03 (1H, m), 2.30-2.37 (1H, m), 2.74-2.81 (1H, m), 3.06 (3H, s), 3.19-3.24 (1H, m), 4.19 (1H, bs), 4.55 (1H, d, J=15.8 Hz), 4.92 (1H, d, J=14.5 Hz), 7.15 (2H, t, J=9.0 Hz), 7.38-7.42 (2H, m), 7.50-7.53 (1H, m), 7.57-7.59 (2H, m), 10.16 (1H, bs), 13.44 (1H, bs). LC-MS (ESI) calcd for C₂₂H₂₂FN₅O₆S₂ 535.1, found 536.4 [M+H⁺].

### EXAMPLE 8

### N-{3-[1-(3,3-Dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶ benzo[1,2,4]thiadiazin-7-yl}-N-methyl-methanesulfonamide

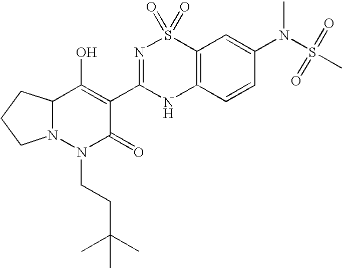

N-{3-[1-(3,3-Dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide (prepared as described in Example 3, 85 mg, 0.16 mmol) was dissolved in N,N-dimethylformamide (6 mL). Potassium carbonate (46 mg, 0.33 mmol) and iodomethane (0.012 mL, 0.18 mmol) were added sequentially. The reaction was stirred at 25° C. for 18 h. The reaction was quenched via the addition of 1.0 M aqueous hydrochloric acid solution (20 mL). The mixture was extracted with ethyl acetate (3×30 mL). The combined organic layers were washed with brine (20 mL), dried over magnesium sulfate, filtered, and concentrated in vacuo to afford a golden oil. Purification by flash column chromatography (Teledyne Isco RediSep Column; 0-20% ethyl acetate in methylene chloride) afforded the desired product, N-{3-[1-(3,3-dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-N-methyl-methanesulfonamide (34 mg, 0.06 mmol, 38%) as a white powder. ¹H NMR (400 MHz, CDCl₃) δ: 1.00 (7.6H, m), 1.46-2.23 (10H), 2.51-2.62 (1.6H, m), 2.88-2.90 (3.5H, m), 2.97 (1.1H, m), 3.10-3.46 (4.6H, m), 3.71-4.01 (2.3H, m), 4.07-4.28 (0.9H, m), 7.18-7.25 (0.8H, m), 7.70-7.81 (1.9H, m), 8.02 (0.6H, s), 13.69 (1.1H, s), 13.91 (1.0H, s). LC-MS (ESI) calcd for C₂₂H₃₁N₅O₆S₂ 525.64, found 526.1 [M+H⁺].

### EXAMPLE 9

### N-{3-[3-(4-Fluoro-benzyl)-6-hydroxy-4-oxo-2,3-diaza-tricyclo[6.2.1.0^{2,7}]undec-5-en-5-yl]-1,1-dioxo-1,4-dihydro-1λ⁶-benzo[1,2,4]thiadiazin-7-yl}-methanesulfonamide

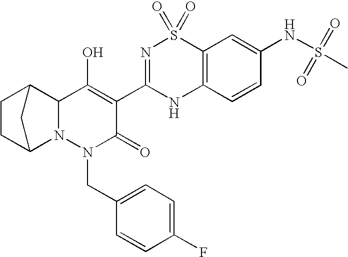

### a) 2-Amino-2-aza-bicyclo[2.2.1]heptane-3-carboxylic acid ethyl ester

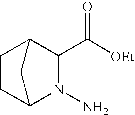

A solution of 2-aza-bicyclo[2.2.1]heptane-3-carboxylic acid ethyl ester hydrochloride (400 mg, 1.94 mmol) in water (10 mL) and treated with saturated aqueous sodium bicarbonate solution and extracted with ethyl acetate (3×10 mL). The combined organic layers were dried over magnesium sulfate, filtered and concentrated in vacuo to afford the free base, 2-aza-bicyclo[2.2.1]heptane-3-carboxylic acid ethyl ester, which was then directly treated with a 0.28 M solution of 1-oxa-2-aza-spiro[2.5]octane in toluene (prepared as described in Example 1g, 14 mL, 3.92 mmol). The reaction mixture was heated at 80° C. for 16 h under a nitrogen atmosphere. After cooling to 0° C., the mixture was extracted with 10% aqueous sulfuric acid solution (1×10 mL, then 2×6 mL). The combined aqueous extracts were neutralized with 1.0 M aqueous sodium hydroxide solution and saturated aqueous sodium bicarbonate solution. The aqueous solution containing the crude product, 2-amino-2-aza-bicyclo[2.2.1]heptane-3-carboxylic acid ethyl ester, was used without any further purification in the next step.

### b) 2-(4-Fluoro-benzylamino)-2-aza-bicyclo[2.2.1]heptane-3-carboxylic acid ethyl ester

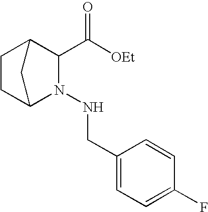

To a solution of 4-fluorobenzaldehyde (215 μL, 2.04 mmol) in methanol heated at 46° C. was added the aqueous solution of crude 2-amino-2-aza-bicyclo[2.2.1]heptane-3-carboxylic acid ethyl ester. After stirring at 46° C. for 3 min, the mixture allowed to cool to 25° C. and stirred for 48 h. The reaction mixture was extracted with chloroform (3×30 mL) and the combined organic layers were dried over magnesium sulfate, filtered, and concentrated in vacuo. The residue was dissolved in acetic acid (20 mL) and sodium cyanoborohydride (305 mg, 4.86 mmol) was added and the mixture was stirred for 48 h. The solvent was removed in vacuo and the residue was dissolved in ethyl acetate (30 mL), washed with saturated aqueous sodium bicarbonate solution (40 mL), brine (40 mL), dried over magnesium sulfate, filtered, and concentrated in vacuo. Purification by flash column chromatography (Teledyne Isco RediSep Column; 0-5% methanol in dichloromethane) afforded the desired product, 2-(4-fluoro-benzylamino)-2-aza-bicyclo[2.2.1]heptane-3-carboxylic acid ethyl ester (270 mg, 0.924 mmol, 47.6% over two steps) as a pale yellow oil. ¹H NMR (400 MHz, CDCl₃) δ: 1.56 (3H, t, J=7.4 Hz), 1.62 (1H, d, J=9.9 Hz), 1.69-1.77 (2H, m), 1.95-2.04 (1H, m), 2.20-2.28 (2H, m), 2.82 (1H, d, J=4.0 Hz), 3.09 (1H, s), 3.91 (1H, s), 4.02 (1H, s), 4.20 (2H, q, J=12.7 Hz), 4.42-4.47 (2H, m), 7.24-7.28 (2H, m), 7.57-7.61 (2H, m). LC-MS (ESI) calcd for C₁₆H₂₁FN₂O₂ 292.16, found 293.2 [M+H⁺].

### c) N-{3-[3-(4-Fluoro-benzyl)-6-hydroxy-4-oxo-2,3-diaza-tricyclo[6.2.1.0^{2,7}]undec-5-en-5-yl]-1,1-dioxo-1,4-dihydro-1λ⁶-benzo[1,2,4]thiadiazin-7-yl}-methanesulfonamide

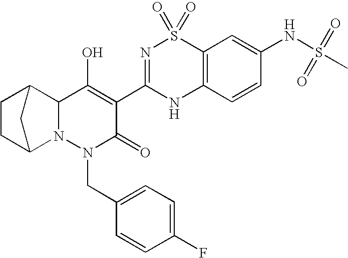

To a solution of 2-(4-fluoro-benzylamino)-2-aza-bicyclo[2.2.1]heptane-3-carboxylic acid ethyl ester (135 mg, 0.462 mmol) and (7-methanesulfonylamino-1,1-dioxo-1,4-dihydro-1λ⁶-benzo[1,2,4]thiadiazin-3-yl)-acetic acid (prepared as described in Example 1f, 186 mg, 0.558 mmol) in N,N-dimethylformamide (5 mL) under an atmosphere of nitrogen, N-(3-dimethylaminopropyl)-N′-ethylcarbodiimide hydrochloride (97 mg, 0.508 mmol) and N-methylmorpholine (127 μL, 1.15 mmol) were added. The reaction mixture was stirred at 25° C. for 16 h. Triethylamine (193 μL, 1.38 mmol) was added and the mixture was heated at 50° C. for 4 h, then additional triethylamine (193 μL, 1.38 mmol) was added and the mixture was heated for another 2 h. Sodium ethoxide (188 mg, 2.76 mmol) was added followed by ethanol (7 mL) and the mixture was heated at 60° C. for 16 h. The reaction mixture was diluted with ethyl acetate (40 mL), washed with 1.0 M aqueous hydrochloric acid solution (2×30 mL), followed by brine (40 mL). The organic layer was dried over magnesium sulfate, filtered, and concentrated in vacuo. The residue was purified by preparative thin-layer chromatography (6% methanol in dichloromethane) to afford the desired product, N-{3-[3-(4-fluoro-benzyl)-6-hydroxy-4-oxo-2,3-diaza-tricyclo[6.2.1.0^{2,7}]undec-5-en-5-yl]-1,1-dioxo-1,4-dihydro-1λ⁶-benzo[1,2,4]thiadiazin-7-yl}-methanesulfonamide (20 mg, 0.036 mmol, 7.7%) as a solid. ¹H NMR (400 MHz, DMSO-d₆) δ: 1.37 (1H, d, J=10.1 Hz), 1.42-1.52 (3H, m), 1.63-1.76 (2H, m), 2.84 (1H, d, J=3.7 Hz), 3.06 (3H, s), 3.63 (1H, bs), 3.98 (1H, bs), 4.41 (1H, d, J=14.7 Hz), 4.97 (1H, d, J=15.8 Hz), 7.12-7.17 (2H, m), 7.35-7.38 (2H, m), 7.49-7.52 (1H, m), 7.54 (1H, s), 7.56 (1H, d, J=2.3 Hz), 10.16 (1H, s), 13.49 (1H, s). LC-MS (ESI) calcd for C₂₄H₂₄FN₅O₆S₂ 561.12, found 562.4 [M+H⁺].

### EXAMPLE 10

### N-[3-(4-Cyclohexyl-7-hydroxy-5-oxo-2,3,3a,4,5,7a,8,8a-octahydro-1H-3b,4-diaza-cyclopenta[alinden-6-yl)-1,1-dioxo-1,4-dihydro-1λ⁶-benzo[1,2,4]thiadiazin-7-yl]-methane sulfonamide

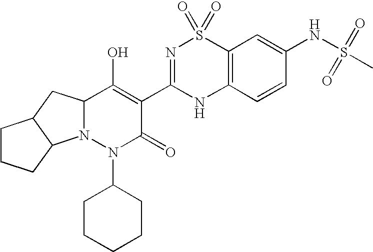

### a) Octahydro-cyclopenta[b]pyrrole-2-carboxylic acid

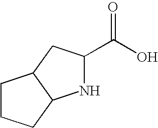

Octahydro-cyclopenta[b]pyrrole-2-carboxylic acid benzyl ester (500 mg, 2.04 mmol) was dissolved in a 1:1 mixture of methanol and ethanol (20 mL) and 10% Palladium on carbon (50 mg) was added. The flask was degassed and backfilled with hydrogen gas via balloon (3×). The mixture was stirred at 25° C. for 4 h, passed through a plug of Celite and rinsed with ethyl acetate. The filtrate was concentrated in vacuo to afford the crude product, octahydro-cyclopenta[b]pyrrole-2-carboxylic acid, which was used directly in the next step without further purification. ¹H NMR (400 MHz, DMSO-d₆) δ: 1.42-1.49 (1H, m), 1.53-1.65 (3H, m), 1.66-1.79 (2H, m), 1.91-1.97 (1H, m), 2.40-2.47 (1H, m), 2.76-2.85 (1H, m), 3.94-3.98 (1H, m), 4.22-4.27 (1H, m). LC-MS (ESI) calcd for C₈H₁₃NO₂ 155.09, found 562.4 [M+H⁺].

### b) Octahydro-cyclopenta[b]pyrrole-2-carboxylic acid methyl ester

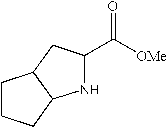

The crude octahydro-cyclopenta[b]pyrrole-2-carboxylic acid was dissolved in a mixture of benzene (15 mL) and methanol (5 mL). The mixture was cooled to 0° C. A 2.0 M solution of (trimethylsilyl)diazomethane in hexanes (1.53 mL, 3.057 mmol) was added and the reaction was stirred at 25° C. for 1 h. The mixture was concentrated in vacuo to afford the desired product, octahydro-cyclopenta[b]pyrrole-2-carboxylic acid methyl ester (300 mg, 1.77 mmol, 86.9% over two steps), as a pale brown solid, which was used directly in the next step without further purification. LC-MS (ESI) calcd for C₉H₁₅NO₂ 169.11, found 170.1 [M+H⁺].

### c) 1-Cyclohexylamino-octahydro-cyclopenta[b]pyrrole-2-carboxylic acid methyl ester

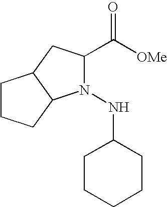

A 0.28 M solution of 1-α-2-aza-spiro[2.5]octane in toluene (prepared as described in Example 1g, 13 mL, 3.64 mmol) was added to the crude octahydro-cyclopenta[b]pyrrole-2-carboxylic acid methyl ester (290 mg, 1.71 mmol) and the mixture was heated at 80° C. for 4 h. The reaction mixture was concentrated in vacuo and the residue was dissolved in glacial acetic acid (15 mL). Cyclohexanone (168 mg, 1.71 mmol) was added and the sodium cyanoborohydride (269 mg, 4.275 mmol) was added in portions at 25° C. The mixture was stirred for 16 h. The solution was concentrated in vacuo and the residue was dissolved in ethyl acetate (40 mL). The organic layer was washed with saturated aqueous sodium bicarbonate solution (30 mL), brine, dried over magnesium sulfate, filtered and concentrated in vacuo. Purification by flash column chromatography (Teledyne Isco RediSep Column; 0-30% ethyl acetate in hexanes) afforded the desired product, 1-cyclohexylamino-octahydro-cyclopenta[b]pyrrole-2-carboxylic acid methyl ester (100 mg, 0.375 mmol, 22%), as an oil. ¹H NMR (400 MHz, CDCl₃) δ: 0.91-1.03 (2H, m), 1.10-1.24 (3H, m), 1.36-1.60 (6H, m), 1.68-1.92 (6H, m), 2.13-2.20 (1H, m), 2.44 (1H, bs), 2.51-2.64 (2H, m), 3.02 (1H, t, J=6.9 Hz), 3.20 (1H, q, J=5.5 Hz), 3.68 (3H, s). LC-MS (ESI) calcd for C₁₅H₂₆N₂O₂ 266.20, found 267.0 [M+H⁺].

### d) 1-{Cyclohexyl-[2-(7-methanesulfonylamino-1,1-dioxo-1,4-dihydro-1λ⁶-benzo[1,2,4]thiadiazin-3-yl)-acetyl]-amino}-octahydro-cyclopenta[b]pyrrole-2-carboxylic acid methyl ester

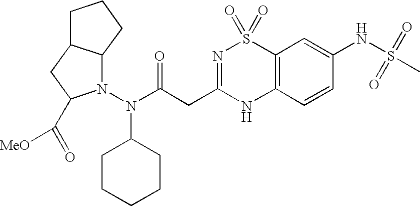

1-Cyclohexylamino-octahydro-cyclopenta[b]pyrrole-2-carboxylic acid methyl ester (100 mg, 0.375 mmol), (7-methanesulfonylamino-1,1-dioxo-1,4-dihydro-1λ⁶-benzo[1,2,4]thiadiazin-3-yl)-acetic acid (prepared as described in Example 1f, 151 mg, 0.413 mmol), and N-(3-dimethylaminopropyl)-N′-ethylcarbodiimide hydrochloride (75 mg, 0.394 mmol) were dissolved in N,N-dimethylformamide (5 mL) under an atmosphere of nitrogen. N-Methylmorpholine (103 μL, 0.938 mmol) was added and the mixture was stirred at 25° C. for 16 h. The mixture was diluted with ethyl acetate (40 mL) and washed with 1.0 M aqueous hydrochloric acid solution (30 mL). The aqueous layer was back-extracted with ethyl acetate (20 mL) and the combined organic layers were washed with brine (40 mL), dried over magnesium sulfate, filtered and concentrated in vacuo. Purification by flash column chromatography (Teledyne Isco RediSep Column; 0-5% methanol in dichloromethane) afforded the desired product, 1-{cyclohexyl-[2-(7-methanesulfonylamino-1,1-dioxo-1,4-dihydro-1λ⁶-benzo[1,2,4]thiadiazin-3-yl)-acetyl]-amino}-octahydro-cyclopenta[b]pyrrole-2-carboxylic acid methyl ester (130 mg, 0.223 mmol, 59.6%), as a yellow oil. LC-MS (ESI) calcd for C₂₅H₃₅N₅O₇S₂ 581.20, found 582.3 [M+H⁺].

### e) N-[3-(4-Cyclohexyl-7-hydroxy-5-oxo-2,3,3a,4,5,7a,8,8a-octahydro-1H-3b,4-diaza-cyclopenta[α] inden-6-yl)-1,1-dioxo-1,4-dihydro-1λ⁶-benzo[1,2,4]thiadiazin-7-yl]-methanesulfonamide

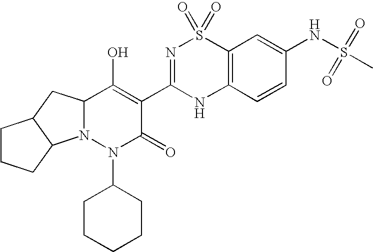

To a solution of 1-{cyclohexyl-[2-(7-methanesulfonylamino-1,1-dioxo-1,4-dihydro-1λ⁶-benzo[1,2,4]thiadiazin-3-yl)-acetyl]-amino}-octahydro-cyclopenta[b]pyrrole-2-carboxylic acid methyl ester (130 mg, 0.223 mmol) in ethanol (10 mL) was added 1,8-diazabicyclo[5,4,0]undec-7-ene (100 μL, 0.67 mmol) and the mixture was heated at 80° C. for 16 h. The mixture was concentrated in vacuo and the residue was dissolved in ethyl acetate (40 mL). The organic layer was washed with 1.0 M aqueous hydrochloric acid solution (10 mL), brine (30 mL), dried over magnesium sulfate, filtered and concentrated in vacuo. The crude product was purified by flash column chromatography (Teledyne Isco RediSep Column; 0-5% methanol in dichloromethane) followed by prep-TLC (5% methanol in dichloromethane) to afford two isomers of the desired product, N-[3-(4-cyclohexyl-7-hydroxy-5-oxo-2,3,3a,4,5,7a,8,8a-octahydro-1H-3b,4-diaza-cyclopenta[a]inden-6-yl)-1,1-dioxo-1,4-dihydro-1λ⁶-benzo[1,2,4]thiadiazin-7-yl]-methanesulfonamide. No attempts were made to assign the isomers. Isomer 1 (60 mg, 0.109 mmol, 49%): ¹H NMR (400 MHz, CDCl₃) δ: 1.03-2.01 (17H, m), 2.17-2.37 (2H, m), 2.65-2.66 (1H, m), 3.05 (3H, s), 3.51 (2H, s), 4.02-4.31 (2H, m), 7.15-7.21 (1H, m), 7.63-7.71 (2H, m), 13.96 (1H, s). LC-MS (ESI) calcd for C₂₄H₃₁N₅O₆S₂, Exact Mass: 549.17, found 550.4 [M+H⁺]. Isomer 2 (30 mg, 0.055 mmol, 24.5%): ¹H NMR (400 MHz, CDCl₃) δ: 0.85-2.21 (19H, m), 2.47-2.72 (2H, m), 3.06 (3H, s), 3.44-4.06 (3H, m), 7.11-7.19 (1H, m), 7.45-7.69 (2H, m), 13.77 (1H, s). LC-MS (ESI) calcd for C₂₄H₃₁N₅O₆S₂, Exact Mass: 549.17, found 550.5 [M+H⁺].

### EXAMPLE 11

### (7S)—N-{3-[3-(4-Fluoro-benzyl)-6-hydroxy-4-oxo-2,3-diaza-tricyclo[6.2.2.0^{2,7}]dodec-5-en-5-yl]-1,1-dioxo-1,4-dihydro-1λ⁶ benzo[1,2,4]thiadiazin-7-yl}-methanesulfonamide

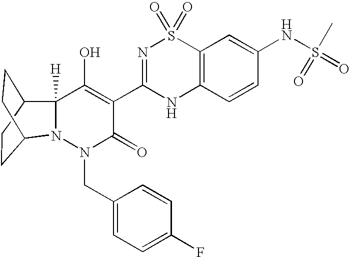

### a) L-Tartaric Acid Dibenzyl Ester

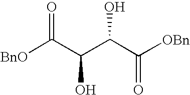

A mixture of L-tartaric acid (3.0 g, 20 mmol), benzyl alcohol (6.5 g, 60 mmol), and p-toluenesulfonic acid (47.5 mg, 0.25 mmol) in toluene (40 mL) was heated in a Dean-Stark apparatus at 130° C. for 16 h. The mixture was allowed to cool to 25° C., diethyl ether was added (50 mL) and the organic layer was washed with saturated aqueous sodium bicarbonate solution (50 mL). The aqueous layer was back-extracted with diethyl ether (2×20 mL) and the combined organic layers were dried over sodium sulfate, filtered and concentrated in vacuo. The residue was triturated with hexanes (200 mL) and diethyl ether (10 mL) to afford the desired product, L-tartaric acid dibenzyl ester (5.86 g, 17.74 mmol, 88.7%), after drying in vacuo at 50° C. for 2 h, as a white solid. LC-MS (ESI) calcd for C₁₈H₁₈O₆ 330.11, found 330.9 [M+H⁺].

### b) Oxo-Acetic Acid Benzyl Ester

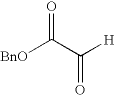

To a solution of L-tartaric acid dibenzyl ester (4.37 g, 13.23 mmol) in diethyl ether (100 mL) was added periodic acid (3.01 g, 13.23 mmol) under an atmosphere of nitrogen. The mixture was stirred at 25° C. for 1.5 h after which time a white precipitate had formed. The mixture was filtered through a plug of Celite and concentrated in vacuo to a volume of ˜3 mL. The crude product, oxo-acetic acid benzyl ester, was used directly in the next step without further purification.

### c) 2-((1R)-Phenyl-ethyl)-2-aza-bicyclo[2.2.2]oct-5-ene-(3S)-carboxylic acid benzyl ester

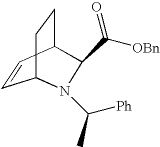

### 2-(1 (R)-Phenyl-ethyl)-2-aza-bicyclo[2.2.2]oct-5-ene-3-carboxylic acid benzyl ester was prepared as described in Tetrahedron Lett. 1996, 37, 7577-7580.

To a solution of oxo-acetic acid benzyl ester (26.46 mmol, crude from Example 11b) in anhydrous dichloromethane at 0° C. was added 4 Å powdered molecular sieves (5.25 g), followed by (R)-(+)-α-methylbenzylamine (3.31 mL, 25.93 mmol). The mixture was allowed to warm to 25° C. and stirred for 48 h. The mixture was filtered through a plug of Celite, rinsed with dichloromethane (50 mL) and the combined filtrates were cooled to −60° C. under an atmosphere of nitrogen. Trifluoroacetic acid (2.04 mL, 26.46 mmol), boron trifluoride-ether complex (3.32 mL, 16.46 mmol) and 1,3-cyclohexadiene (2.77 mL, 29.10 mmol) were added sequentially. The reaction mixture was stirred for 3 h at −60° C., then for 16 h at −25° C. Upon warming to 25° C., saturated aqueous sodium bicarbonate solution (100 mL) was added and the mixture was stirred for 1 h. The layers were separated and the aqueous layer was extracted with dichloromethane (2×50 mL). The combined organic layers were washed with saturated aqueous sodium bicarbonate solution (100 mL), dried over magnesium sulfate, filtered and concentrated in vacuo. The crude product was purified by flash column chromatography (Teledyne Isco RediSep Column; 0-20% ethyl acetate in hexanes) to afford the desired product, 2-((1R)-phenyl-ethyl)-2-aza-bicyclo[2.2.2]oct-5-ene-(3S)-carboxylic acid benzyl ester (2.73 g, 7.86 mmol, 29.7%). ¹H NMR (400 MHz, CDCl₃) δ: 0.95-1.01 (1H, m), 1.18-1.33 (3H, m), 1.51-1.62 (2H, m), 1.98-2.06 (1H, m), 2.71 (1H, bs), 2.93 (1H, bs), 3.38-3.42 (1H, m), 3.60 (1H, bs), 4.90 (2H, s), 6.20-6.24 (1H, m), 6.32-6.36 (1H, m), 7.11-7.35 (10H, m).

### d) 2-Aza-bicyclo[2.2.2]octane-(3S)-carboxylic acid

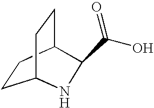

To a solution of 2-((1R)-phenyl-ethyl)-2-aza-bicyclo[2.2.2]oct-5-ene-(3S)-carboxylic acid benzyl ester (2.73 g, 7.86 mmol) in methanol (70 mL) was added 5% Pd—C (50% in water, 1 g). The mixture was hydrogenated under a hydrogen atmosphere (45 psi, Parr shaker) for 2 h. The mixture was filtered through Celite and concentrated in vacuo. The resulting solid was suspended in diethyl ether (50 mL), sonicated and collected by vacuum filtration. The solid was washed with diethyl ether (80 mL) and dried in vacuo at 50° C. for 16 h to afford the desired product, 2-aza-bicyclo[2.2.2]octane-(3S)-carboxylic acid (1.17 g, 7.54 mmol, 96%), as an off-white solid.

### e) 2-Aza-bicyclo[2.2.2]octane-(3S)-carboxylic acid methyl ester

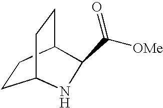

2-Aza-bicyclo[2.2.2]octane-(3S)-carboxylic acid (1.10 g, 7.09 mmol) was suspended in benzene (30 mL) and methanol (10 mL) and the mixture was cooled to 0° C. A 2.0 M solution of (trimethylsilyl)diazomethane in hexanes (4.25 mL, 8.5 mmol) was added and the reaction was stirred at 0° C. for 1 h and then at 25° C. for 1 h. The mixture was concentrated in vacuo to afford the crude product, 2-aza-bicyclo[2.2.2]octane-(3S)-carboxylic acid methyl ester (1.0 g, 5.91 mmol, 83.4% crude), as a solid, which was used directly in the next step without further purification. LC-MS (ESI) calcd for C₉H₁₅NO₂ 169.11, found 170.1 [M+H⁺].

### f) 2-Amino-2-aza-bicyclo[2.2.2]octane-(3S)-carboxylic acid methyl ester

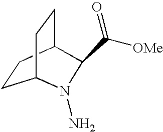

A 0.28 M solution of 1-oxa-2-aza-spiro[2.5]octane in toluene (prepared as described in Example 1g, 21 mL, 5.88 mmol) was added to 2-aza-bicyclo[2.2.2]octane-(3S)-carboxylic acid methyl ester (485 mg, 2.87 mmol) and the mixture was heated at 80° C. for 17 h. Upon cooling to 25° C., the reaction mixture was extracted with an ice-cold 10% aqueous sulfuric acid solution (3×10 mL). The combined cold aqueous layers were neutralized with saturated aqueous sodium bicarbonate solution (60 mL). This solution containing the crude product, 2-amino-2-aza-bicyclo[2.2.2]octane-(3S)-carboxylic acid methyl ester, was used directly in the next step without further purification. LC-MS (ESI) calcd for C₉H₁₆N₂O₂ 184.12, found 184.1 [M⁺].

### g) 2-(4-Fluoro-benzylamino)-2-aza-bicyclo[2.2.2]octane-(3S)-carboxylic acid methyl ester

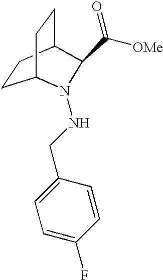

To a solution of 4-fluorobenzaldehyde (309 μL, 2.87 mmol) in methanol (36 mL) at 45° C., was added 2-amino-2-aza-bicyclo[2.2.2]octane-(3S)-carboxylic acid methyl ester (crude from Example 11f, 2.87 mmol). After stirring at 45° C. for 2 h, the solution was allowed to cool to 25° C. and was stirred at that temperature for 16 h. The mixture was extracted with chloroform (3×40 mL) and the combined extracts were washed with brine (40 mL), dried over magnesium sulfate, filtered, and concentrated in vacuo. The residue was dissolved in glacial acetic acid (10 mL) and sodium cyanoborohydride (360 mg, 5.73 mmol) was added. After stirring for 3 h at 25° C. the mixture was concentrated in vacuo. The residue was dissolved in ethyl acetate (40 mL), washed with saturated aqueous sodium bicarbonate solution (30 mL), dried over magnesium sulfate, filtered and concentrated in vacuo. Purification by flash column chromatography (Teledyne Isco RediSep Column; 0-5% methanol in dichloromethane) afforded the desired product, 2-(4-fluoro-benzylamino)-2-aza-bicyclo[2.2.2]octane-(3S)-carboxylic acid methyl ester (250 mg, 0.856 mmol, 29.8%), as a pale yellow oil. ¹H NMR (400 MHz, CDCl₃) δ: 1.27-2.06 (10H, m), 2.99 (1H, bs), 3.41 (1H, bs), 3.73 (3H, s), 3.94 (2H, s), 6.97-7.01 (2H, m), 7.30-7.33 (2H, m). LC-MS (ESI) calcd for C₁₆H₂₁FN₂O₂ 292.16, found 293.1 [M+H⁺].

### h) (7S)—N-{3-[3-(4-Fluoro-benzyl)-6-hydroxy-4-oxo-2,3-diaza-tricyclo[6.2.2.02]dodec-5-en-5-yl]-1,1-dioxo-1,4-dihydro-1λ⁶-benzo[1,2,4]thiadiazin-7-yl}-methanesulfonamide

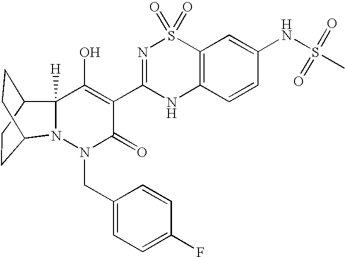

2-(4-Fluoro-benzylamino)-2-aza-bicyclo[2.2.2]octane-(3S)-carboxylic acid methyl ester (250 mg, 0.856 mmol), (7-methanesulfonylamino-1,1-dioxo-1,4-dihydro-1λ⁶-benzo[1,2,4]thiadiazin-3-yl)-acetic acid (prepared as described in Example 1f, 344 mg, 0.941 mmol), and N-(3-dimethylaminopropyl)-N′-ethylcarbodiimide hydrochloride (180 mg, 0.941 mmol) were dissolved in N,N-dimethylformamide (5 mL) under an atmosphere of nitrogen. N-Methylmorpholine (235 μL, 2.14 mmol) was added and the mixture was stirred at 25° C. for 60 h.

1,8-diazabicyclo[5,4,0]undec-7-ene (391 mg, 2.568 mmol) was added and the mixture was heated at 80° C. for 2 h. The mixture was diluted with ethyl acetate (50 mL) and washed with 1.0 M aqueous hydrochloric acid solution (50 mL). The aqueous layer was back-extracted with ethyl acetate (30 mL) and the combined organic layers were washed with brine (3×30 mL), dried over magnesium sulfate, filtered and concentrated in vacuo. The residue was dissolved in anhydrous methanol (20 mL), 1,8-diazabicyclo[5,4,0]undec-7-ene (391 mg, 2.568 mmol) was added and the mixture was heated at 80° C. for 16 h. The mixture was concentrated in vacuo and the residue was dissolved in ethyl acetate (50 mL). The organic layer was washed with 1.0 M aqueous hydrochloric acid solution (40 mL), brine, dried over magnesium sulfate, filtered and concentrated in vacuo. The residue was dissolved in hot ethyl acetate and a first batch of the product (61 mg) was precipitated with hexanes. A second batch was further purified by flash column chromatography (Teledyne Isco RediSep Column; 0-5% methanol in dichloromethane) to afford an additional batch of the desired product, (7S)—N-{3-[3-(4-fluoro-benzyl)-6-hydroxy-4-oxo-2,3-diaza-tricyclo[6.2.2.0^{2,7}]dodec-5-en-5-yl]-1,1-dioxo-1,4-dihydro-1λ⁶-benzo[1,2,4]thiadiazin-7-yl}-methanesulfonamide (70 mg; total yield: 131 mg, 0.228 mmol, 26.6%) as a pale yellow solid. ¹H NMR (400 MHz, DMSO-d₆) δ: 1.34-1.52 (2H, m), 1.54-1.78 (4H, m), 1.82-1.90 (1H, m), 1.96-2.08 (1H, m), 2.25 (1H, bs), 3.06 (3H, s), 3.10 (1H, bs), 4.17 (1H, bs), 4.30 (1H, d, J=15.6 Hz), 5.00 (1H, d, J=15.0 Hz), 7.12-7.17 (2H, m), 7.36-7.39 (2H, m), 7.50-7.53 (1H, m), 7.58-7.60 (2H, m), 10.17 (1H, s), 13.32 (1H, s). LC-MS (ESI) calcd for C₂₅H₂₆FN₅O₆S₂ 575.13, found 576.4 [M+H⁺].

### EXAMPLE 12

### (1aR,7aS)—N-{3-[4-(4-Fluoro-benzyl)-7-hydroxy-5-oxo-1,1a,2,3,4,5-hexahydro-3a,4-diaza-cyclopropa[c]inden-6-yl]-1,1-dioxo-1,4-dihydro-1λ⁶-benzo[1,2,4]thiadiazin-7-yl}-methanesulfonamide

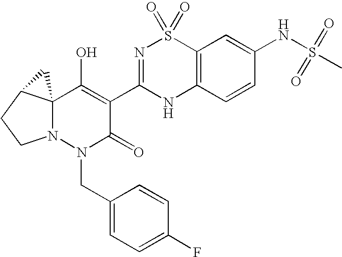

### a) (1S,5R)-2-Amino-2-aza-bicyclo[3.1.0]hexane-1-carboxylic acid methyl ester

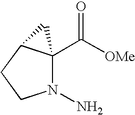

A 0.28 M solution of 1-oxa-2-aza-spiro[2.5]octane in toluene (prepared as described in Example 1g, 13 mL, 3.64 mmol) was added to 2-aza-bicyclo[3.1.0]hexane-(1S)-carboxylic acid methyl ester (260 mg, 1.84 mmol) and the mixture was heated at 80° C. for 17 h. Upon cooling to 25° C., the reaction mixture was extracted with ice-cold 10% aqueous sulfuric acid solution (3×10 mL). The combined cold aqueous layers were neutralized with saturated aqueous sodium bicarbonate solution (60 mL). This solution containing the crude product, (1S,5R)-2-amino-2-aza-bicyclo[3.1.0]hexane-1-carboxylic acid methyl ester, was used directly in the next step without further purification.

### b) (1S,5R)-2-(4-Fluoro-benzylamino)-2-aza-bicyclo[3.1.0]hexane-1-carboxylic acid methyl ester

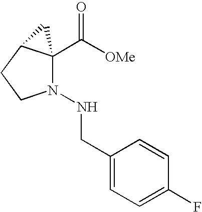

To a solution of 4-fluorobenzaldehyde (198 μL, 1.84 mmol) in methanol (25 mL) at 45° C., was added (1S,5R)-2-amino-2-aza-bicyclo[3.1.0]hexane-1-carboxylic acid methyl ester (crude from Example 12a, 1.84 mmol). The reaction mixture was stirred at 25° C. for 16 h. The mixture was extracted with chloroform (3×40 mL) and the combined extracts were washed with brine, dried over magnesium sulfate, filtered, and concentrated in vacuo. The residue was dissolved methanol (10 mL), trifluoroacetic acid (1 mL) and sodium cyanoborohydride (231 mg, 3.68 mmol) was added. After stirring for 2 h at 25° C. the mixture was concentrated in vacuo. The residue was dissolved in ethyl acetate (50 mL), washed with saturated aqueous sodium bicarbonate solution, dried over magnesium sulfate, filtered and concentrated in vacuo. Purification by flash column chromatography (Teledyne Isco RediSep Column; 0-7% methanol in dichloromethane) afforded the desired product, (1S,5R)-2-(4-fluoro-benzylamino)-2-aza-bicyclo [3.1.0]hexane-1-carboxylic acid methyl ester (320 mg, 1.21 mmol, 65.8%) as a yellow oil. ¹H NMR (400 MHz, CDCl₃) δ: 1.39-1.45 (2H, m), 1.89-1.95 (2H, m), 2.06-2.22 (2H, m), 3.47 (1H, t, J=8.6 Hz), 3.75 (3H, s), 4.05 (2H, s), 6.98-7.02 (2H, m), 7.32-7.35 (2H, m). LC-MS (ESI) calcd for C₁₄H₁₇FN₂O₂ 264.13, found 265.1 [M+H⁺].

### c) (1aR,7aS)—N-{3-[4-(4-Fluoro-benzyl)-7-hydroxy-5-oxo-1,1a,2,3,4,5-hexahydro-3a,4-diaza-cyclopropa[c]inden-6-yl]-1,1-dioxo-1,4-dihydro-1λ⁶-benzo[1,2,4]thiadiazin-7-yl}-methanesulfonamide

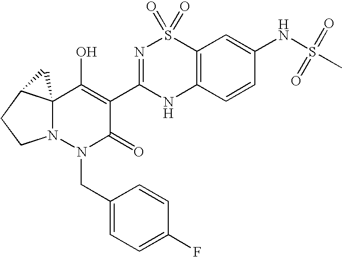

(1S,5R)-2-(4-Fluoro-benzylamino)-2-aza-bicyclo[3.1.0]hexane-1-carboxylic acid methyl ester (160 mg, 0.60 mmol), (7-methanesulfonylamino-1,1-dioxo-1,4-dihydro-1λ⁶-benzo[1,2,4]thiadiazin-3-yl)-acetic acid (prepared as described in Example 1f, 244 mg, 0.666 mmol), and N-(3-dimethylaminopropyl)-N′-ethylcarbodiimide hydrochloride (128 mg, 0.666 mmol) were dissolved in N,N-dimethylformamide (5 mL) under an atmosphere of nitrogen. N-Methylmorpholine (166 μL, 1.51 mmol) was added and the mixture was stirred at 25° C. for 16 h. 1,8-Diazabicyclo[5,4,0]undec-7-ene (274 mg, 1.8 mmol) was added and the mixture was heated at 80° C. for 23 h. Upon cooling to 25° C., the mixture was diluted with ethyl acetate (50 mL) and washed with 1.0 M aqueous hydrochloric acid solution (50 mL). The aqueous layer was back-extracted with ethyl acetate (40 mL) and the combined organic layers were washed with brine (2×40 mL), dried over magnesium sulfate, filtered and concentrated in vacuo. The residue was purified by flash column chromatography (Teledyne Isco RediSep Column; 0-7% methanol in dichloromethane) to afford the desired product, (1aR,7aS)—N-{3-[4-(4-fluoro-benzyl)-7-hydroxy-5-oxo-1,1a,2,3,4,5-hexahydro-3a,4-diaza-cyclopropa[c]inden-6-yl]-1,1-dioxo-1,4-dihydro-1λ⁶-benzo[1,2,4]thiadiazin-7-yl}-methanesulfonamide (103 mg, 0.188 mmol, 31.4%) as a yellow solid. ¹H NMR (400 MHz, DMSO-d₆) δ: 1.23-1.26 (1H, m), 1.47-1.55 (1H, m), 2.01-2.05 (1H, m), 2.08-2.14 (1H, m), 2.41-2.46 (1H, m), 3.06 (3H, s), 3.47-3.50 (2H, m), 4.48 (1H, d, J=15.4 Hz), 4.99 (1H, d, J=15.7 Hz), 7.13-7.18 (2H, m), 7.34-7.37 (2H, m), 7.50-7.59 (3H, m), 10.16 (1H, s), 13.44 (1H, s). LC-MS (ESI) calcd for C₂₃H₂₂FN₅O₆S₂ 547.10, found 548.3 [M+H⁺].

### Biological Testing

The ability of compounds of Formula I to inhibit HCV replication can be demonstrated in the following in vitro assays.

Compounds were tested for HCV polymerase inhibition. Assays were performed in a 96-well streptavidin-coated FlashPlate using 20 nM enzyme, 0.5 μCi of [α-³³P]GTP, 0.6 μM GTP, and 250 nM 5′biotinylated oligo (rG₁₃)/poly rC in 20 mM Tris-HCl, pH 7.5, 5 mM MgCl₂, 5 mM dithiothreitol, 0.1 g/L bovine serum albumin, and 100 U/mL RNAse inhibitor. The reaction was stopped by aspiration after 75 min at 28° C. and the plate was washed several times. After washing and drying the plate, incorporated radioactivity was counted using a Microbeta scintillation counter. IC₅₀ values were calculated relative to the uninhibited control and inhibition data were fitted to a 4-parameter IC₅₀ equation. For very potent inhibitors, the data were fitted to a tight binding quadratic equation to obtain IC₅₀ values.

Test results (IC₅₀ values) for compounds of Formula I are summarized in Table 1, wherein +++ means NS5B polymerase inhibition with IC₅₀ values less than 0.02 μM, ++ means IC₅₀ values between 0.02 μM and 0.2 μM, and + means IC₅₀ values between 0.2 μM and 3 μM.

TABLE 1

| Example # | Structure | Name | IC50 |
|---|---|---|---|
| 1 | 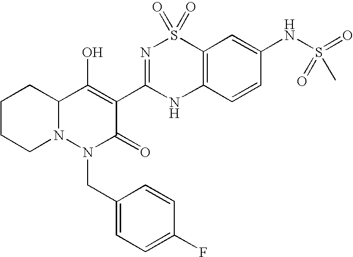 | N-{3-[1-(4-Fluoro-benzyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methanesulfonamide | +++ |
| 2 | 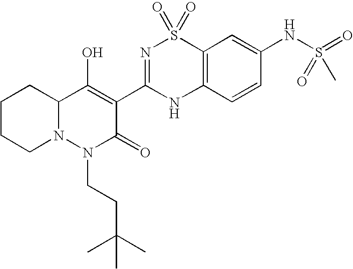 | N-{3-[1-(3,3-Dimethyl-butyl)-4-hydroxy-2-oxo-2,4a,5,6,7,8-hexahydro-1H-pyrido[1,2-b]pyridazin-3-yl]-1,1-dioxo-1,4-dihydro-1λ⁶-benzo[1,2,4]thiadiazin-7-yl}-methanesulfonamide | ++ |
| 3 | 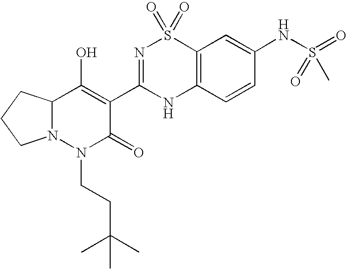 | N-{3-[1-(3,3-Dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide | +++ |
| 4 | 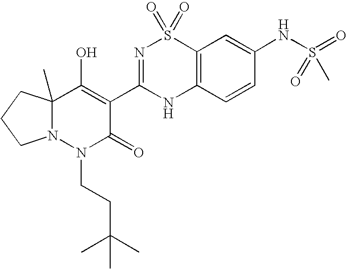 | N-{3-[1-(3,3-Dimethyl-butyl)-4-hydroxy-4a-methyl-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide | ++ |
| 5 | 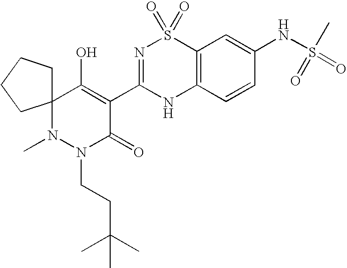 | N-{3-[7-(3,3-Dimethyl-butyl)-10-hydroxy-6-methyl-8-oxo-6,7-diaza-spiro[4.5]dec-9-en-9-yl]-1,1-dioxo-1,4-dihydro-1λ⁶-benzo[1,2,4]thiadiazin-7-yl}-methanesulfonamide | + |
| 6 | 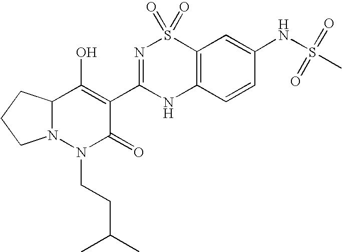 | N-{3-[4-Hydroxy-1-(3-methyl-butyl)-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide | +++ |
| 7 | 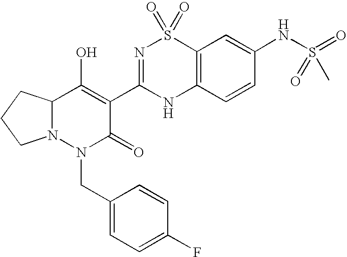 | N-{3-[1-(4-Fluoro-benzyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-methanesulfonamide | +++ |
| 8 | 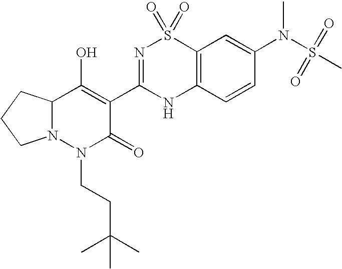 | N-{3-[1-(3,3-Dimethyl-butyl)-4-hydroxy-2-oxo-1,2,4a,5,6,7-hexahydro-pyrrolo[1,2-b]pyridazin-3-yl]-1,1-dioxo-1,4-dihydro-1λ⁶-benzo[1,2,4]thiadiazin-7-yl}-N-methyl-methanesulfonamide | ++ |
| 9 | 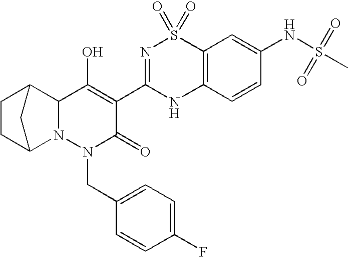 | N-{3-[3-(4-Fluoro-benzyl)-6-hydroxy-4-oxo-2,3-diaza-tricyclo[6.2.1.0^{2,7}]undec-5-en-5-yl]-1,1-dioxo-1,4-dihydro-1λ⁶-benzo[1,2,4]thiadiazin-7-yl}-methanesulfonamide | +++ |
| 10 | 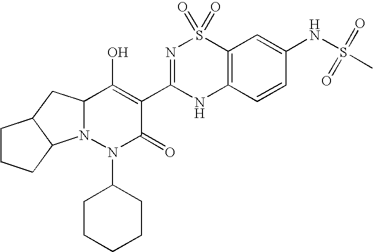 | N-[3-(4-Cyclohexyl-7-hydroxy-5-oxo-2,3,3a,4,5,7a,8,8a-octahydro-1H-3b,4-diaza-cyclopenta[a]inden-6-yl)-1,1-dioxo-1,4-dihydro-1λ⁶-benzo[1,2,4]thiadiazin-7-yl]-methanesulfonamide | Isomer 1: +Isomer 2: + |
| 11 | 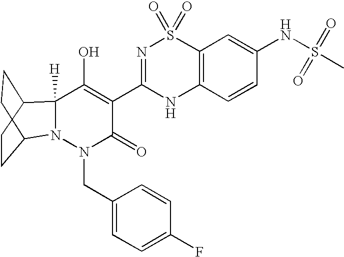 | (7S)-N-{3-[3-(4-Fluoro-benzyl)-6-hydroxy-4-oxo-2,3-diaza-tricyclo[6.2.2.0^{2,7}]dodec-5-en-5-yl]-1,1-dioxo-1,4-dihydro-1λ⁶-benzo[1,2,4]thiadiazin-7-yl}-methanesulfonamide | ++ |
| 12 | 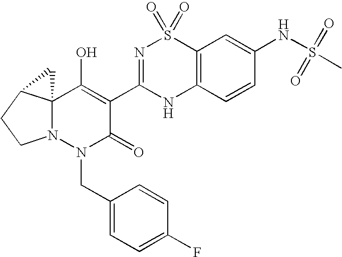 | (1aR,7aS)-N-{3-[4-(4-Fluoro-benzyl)-7-hydroxy-5-oxo-1,1a,2,3,4,5-hexahydro-3a,4-diaza-cyclopropa[c]inden-6-yl]-1,1-dioxo-1,4-dihydro-1λ⁶-benzo[1,2,4]thiadiazin-7-yl}-methanesulfonamide | ++ |

### HCV Replicon Assay (Replicon EC₅₀ (μM))

The cell culture component of the assay is performed essentially as described by Bartenschlager et al., Hepatology 2002, 35, 694-703, wherein exponentially growing HCV Huh-7/C24 replicon cells are seeded at 4.5×10³ cells/well in 96 well plates and 24 hours later are treated with six point half-log concentration of compound. After 72 hours exposure the media is discarded from the compound assay plate and the cell monolayers are lysed by addition of 150 □l lysis mixture (Genospectra) with incubation at 53° C. for 45 minutes. Following incubation, each lysate is thoroughly mixed and 5 □l (NS3 probe) or 10 □l (GAPDH probe) of each lysate is then transferred to the capture plate and analyzed by bDNA assay.

Branched DNA (bDNA) Assay

Based on provided sequences for NS3 [AJ242652], Genospectra (Fremont, Calif., USA) designed and synthesized probes to these analytes (together with GAPDH). Cellular bDNA analysis is carried out essentially as described in the Genospectra protocol (details in Shyamala, V. et al., Anal Biochem 1999, 266, 140-7), wherein target specific capture extenders, label extenders and blocking probes are added to the capture plate after the addition of 5 or 10 μl cell lysate. After annealing overnight, during which the target RNA is captured to the plate via interaction with the capture extenders, the plate is washed, and then amplifier (which binds via the label extenders) and label probe are sequentially added.

After subsequent addition of the chemilumigenic substrate (dioxetan), each plate is read by luminometer (Wallac 1420 Multilabel HTS Counter Victor 2). The luminescence signal is proportional to the amount of mRNA present in each lysate. In addition to the samples, cell lysate only (no probe) background controls are also included on each bDNA assay plate and the average signal from these control wells is subtracted from the sample reading prior to analysis. Percent of no drug control is determined for both the NS3 and GAPDH signals for each compound also. Percent inhibition is determined for each compound concentration in relation to the no drug control to calculate the EC₅₀.

It is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention.

What is claimed is:

1. A compound of Formula I 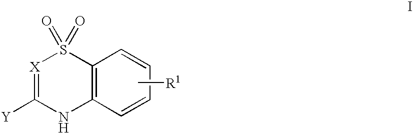
wherein
X is N or CR³,
Y is 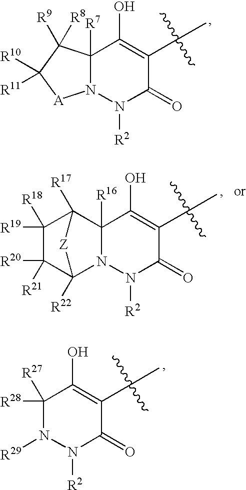
wherein
A is —CR¹²R¹³— or —CR¹²R¹³—CR¹⁴R¹⁵—,
Z is —CR²³R²⁴— or —CR²³R²⁴—CR²⁵R²⁶—,
R¹ is H, halo, nitro, —CHR⁴—S(O)₂R⁵, —NR⁵R⁶, —NR⁴S(O)₂R⁵, or —NR⁴S(O)₂NR⁵R⁶, wherein R⁴, R⁵, and R⁶ are independently H, C₁-C₆ alkyl, C₃-C₈ cycloalkyl, aryl, or heterocyclyl, or R⁴ and R⁵ or R⁵ and R⁶ combine with the atom(s) to which they are attached to form a 5- or 6-membered heterocyclyl ring,
R² is C₁-C₆ alkyl, C₃-C₈ cycloalkyl, —C₁-C₆ alkylene(C₃-C₈ cycloalkyl), —C₁-C₆ alkylene(aryl), or —C₁-C₆ alkylene(heterocyclyl),
R³ is H, halo, or C₁-C₆ alkyl,
R⁷ is H, C₁-C₆ alkyl, or C₃-C₈ cycloalkyl, or R⁷ and R⁸ or R⁷ and R⁹ combine to form a 3-membered ring,
R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently H, C₁-C₆ alkyl, or halo, or
R¹⁰ and R¹² or R¹⁰ and R¹³, or R¹¹ and R¹² or R¹¹ and R¹³ combine to form a 5 membered ring, or
R²⁷ and R²⁸ combine to form a 3- to 5-membered ring,
R²⁹ is H or C₁-C₆ alkyl,
wherein the above alkyl, alkylene, aryl, cycloalkyl, or heterocyclyl moieties provided in R¹ through R²⁹ are each optionally and independently substituted by 1-3 substituents selected from
alkylamine,
amino,
aryl, cycloalkyl, heterocyclyl,
C₁-C₆ alkyl, C₁-C₆ haloalkyl, C₁-C₆ hydroxyalkyl, C₁-C₆ alkoxy, C₁-C₆ alkylamine, C₁-C₆ dialkylamine, C₂-C₆ alkenyl, or C₂-C₆ alkynyl, wherein each of which may be interrupted by one or more hetero atoms,
carboxyl,
cyano,
halo,
hydroxy,
nitro,
—C(O)OH, —C(O)₂—(C₁-C₆ alkyl), —C(O)₂—(C₃-C₈ cycloalkyl), —C(O)₂-(aryl), —C(O)₂-(heterocyclyl), —C(O)₂—(C₁-C₆ alkylene)aryl, —C(O)₂—(C₁-C₆ alkylene)heterocyclyl, —C(O)₂—(C₁-C₆ alkylene)cycloalkyl, —C(O)(C₁-C₆ alkylene), —C(O)(C₃-C₈ cycloalkyl), —C(O)(aryl), —C(O)(heterocyclyl), —C(O)(C₁-C₆ alkylene)aryl, —C(O)(C₁-C₆ alkylene)heterocyclyl, and —C(O)(C₁-C₆ alkyl)cycloalkyl,
wherein each of the above optional substituents can be further optionally substituted by 1-5 substituents selected from amino, cyano, halo, hydroxy, nitro, C₁-C₆ alkylamine, C₁-C₆ dialkylamine, C₁-C₆ alkyl, C₁-C₆ alkoxy, C₁-C₆ alkenyl, and C₁-C₆ hydroxyalkyl, wherein each alkyl is optionally substituted by one or more halo substituents,
or a pharmaceutically acceptable salt, hydrate, tautomer or stereoisomer thereof.

2. The compound according to claim 1 wherein R¹ is —NR⁴S(O)₂R⁵, wherein R⁴ and R⁵ are independently H or C₁-C₆ alkyl.

3. The compound according to claim 2 wherein R¹ is selected from

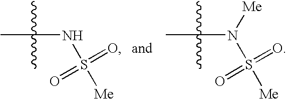

4. The compound according to claim 1 wherein R² is selected from

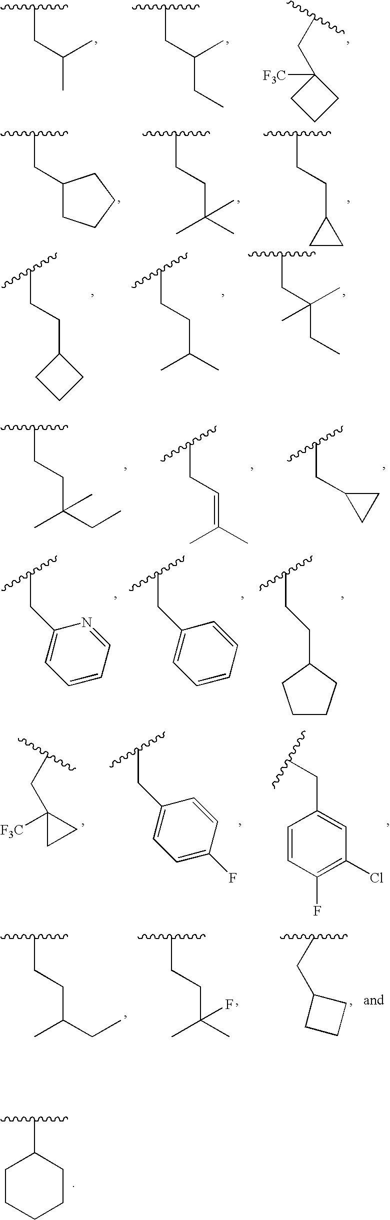

5. The compound according to claim 4 wherein R² is selected from

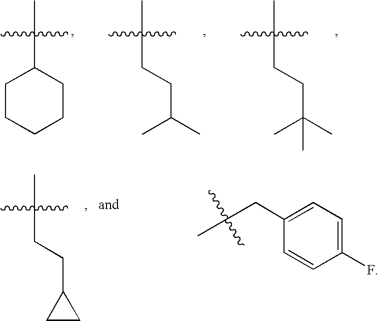

6. The compound of claim 5 wherein R² is selected from

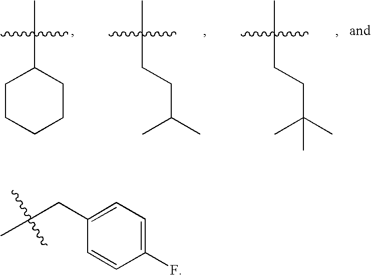

7. The compound of claim 1 wherein R³ is selected from

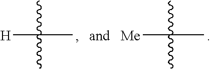

8. The compound according to claim 1 wherein X is N.

9. The compound according to claim 1 wherein R⁷ is selected from H or C₁-C₆ alkyl.

10. The compound according to claim 9 wherein R⁷ is selected from

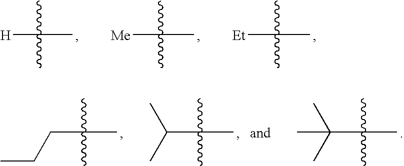

11. The compound according to claim 10 wherein R⁷ is selected from

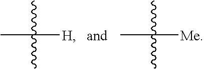

12. The compound according to claim 1 wherein Y is 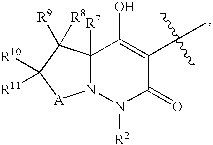
wherein A is —CR¹²R¹³.

13. The compound according to claim 1 wherein R⁸, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from

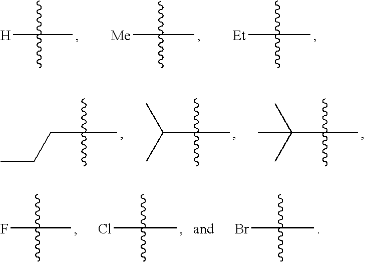

14. The compound according to claim 13 wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are independently selected from

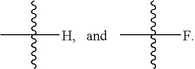

15. The compound according claim 14 wherein R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ are H.

16. The compound according to claim 1 wherein R²⁹ is methyl.

17. A compound selected from

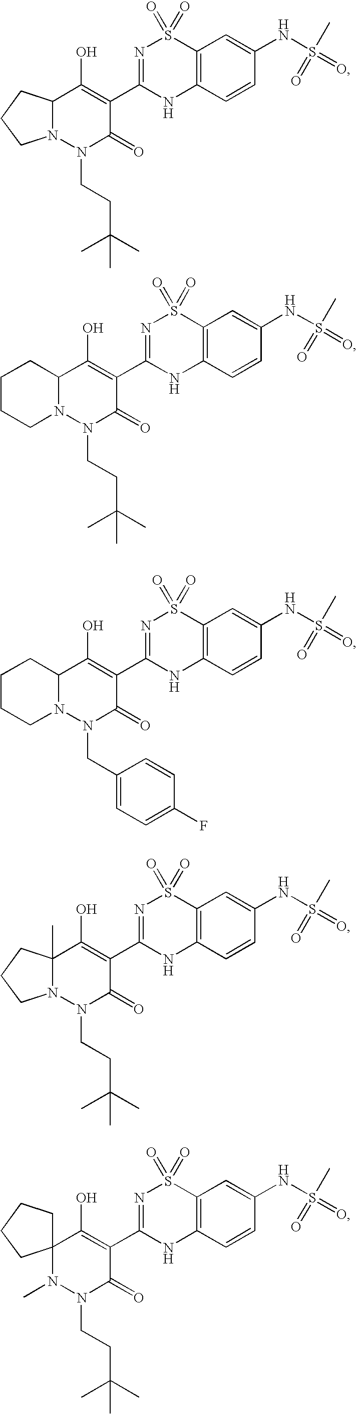

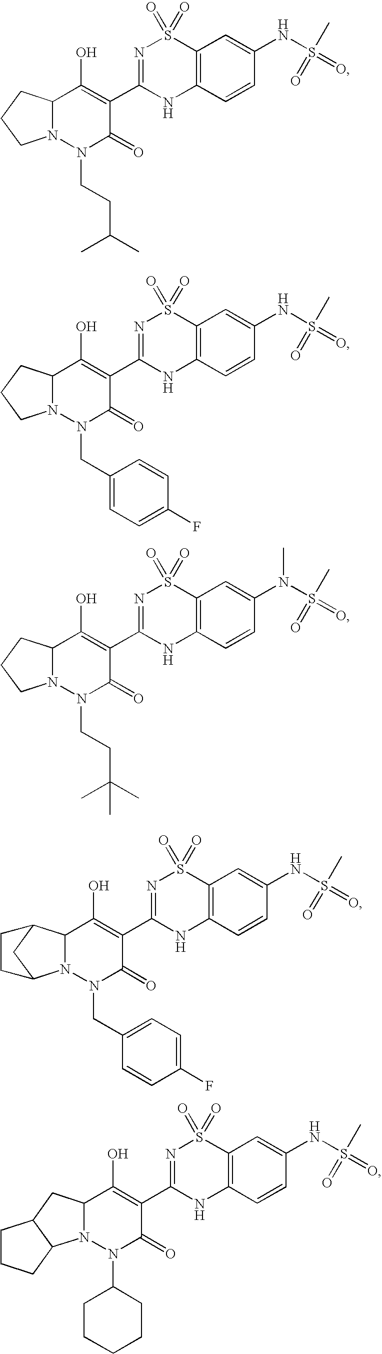

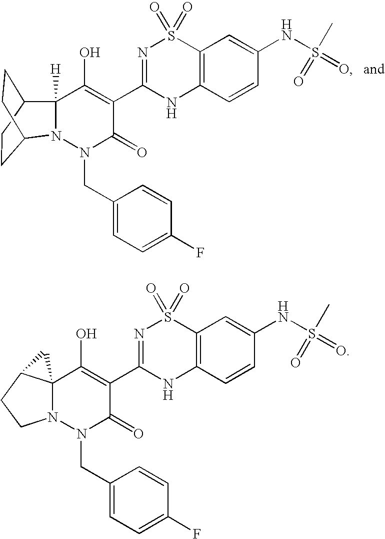

18. A pharmaceutically acceptable composition comprising a compound of claim 1 and a pharmaceutically acceptable carrier.

19. A method of inhibiting hepatitis C virus replication comprising exposing hepatitis C virus to a therapeutically effective concentration of a compound of claim 1.

20. A method for treating or preventing hepatitis C virus infection in a mammal in need thereof, comprising administering to the mammal a therapeutically or prophylactically effective amount of a compound of claim 1.

21. The method of claim 20 wherein the mammal is a human.

22. The method of claim 20 further comprising administering an additional therapeutic agent to the mammal.

23. The method of claim 22 wherein the additional therapeutic agent is selected from the group consisting of an antibiotic, an antiemetic agent, an antidepressant, an antifungal agent, an anti-inflammatory agent, an antiviral agent, an anticancer agent, an immunomodulatory agent, an α-interferon, a β-interferon, a ribavirin, an alkylating agent, a hormone, a cytokine and a toll receptor-like modulator.
